SEQUENCE LISTING (2 sequences)

SEQ ID NO: 1 - DNA, 13 bp, Homo sapiens
agaggttgagtcc

SEQ ID NO: 2 - DNA, 17 bp, Homo sapiens
agaggttctttgagtcc

(19) United States
(12) Patent Application Publication
(10) Pub. No.: US 2010/0158868 A1
(43) Pub. Date: Jun. 24, 2010

(54) Use of Fetal Cells for the Treatment of Genetic Diseases

(75) Inventor: Yuet W. Kan, San Francisco, CA (US)

Correspondence Address: MORGAN, LEWIS & BOCKIUS, LLP (SF), ONE MARKET SPEAR STREET TOWER, SAN FRANCISCO, CA, 94105, US

(73) Assignee: Yuet W. Kan, San Francisco, CA (US)

(21) Appl. No.: 12/643,639

(22) Filed: Dec. 21, 2009

Related U.S. Application Data
(60) Provisional application No. 61/266,876, filed on Dec. 4, 2009.
(60) Provisional application No. 61/139,389, filed on Dec. 19, 2008.

(51) Int. Cl. A61K 48/00; C12N 5/02; C12N 5/071; C12N 15/63

(52) U.S. Cl. 424/93.2; 435/377; 435/366; 435/455

(57) ABSTRACT

The present invention provides iPS and stem cells derived from fetal somatic cells derived from a fetus in utero or from cord blood. The iPS and stem cells can be stored or used to treat genetic diseases.

### RELATED APPLICATION

This application claims the benefit of, and priority to, U.S. Provisional Patent Application Ser. No. 61/266,876 filed Dec. 4, 2009, entitled “Use of Fetal Cells for the Treatment of Genetic Diseases”, and U.S. Provisional Patent Application Ser. No. 61/139,389 filed Dec. 19, 2008, entitled “Use of Fetal Cells for the Treatment of Genetic Diseases”, the disclosure of which is hereby incorporated by reference in its entirety.

### BACKGROUND OF THE INVENTION

The withdrawal of amniotic fluid, a procedure known as amniocentesis, has been practiced for the past 100 years, and in the past 50 years, the cells in the fluid have been used for prenatal diagnosis of inherited genetic diseases such as Down syndrome, cystic fibrosis, sickle cell anemia or thalassemia. If the fetus is found to be affected by a severe genetic disease such as sickle cell anemia or thalassemia, the parents would have to make a decision whether to terminate the pregnancy, or continue it and care for the child with severe lifelong illness. The cells that have been used for the diagnosis served no further purpose and were either discarded or stored for a variable period in case the diagnosis needs to be verified.

Human embryonic stem (ES) cells have been proposed for the treatment of various diseases. Histoincompatibilty is a significant potential complication with human ES cells obtained from unrelated donors. Patient specific embryonic stem cells require nuclear transfer which is technically challenging and not yet achievable in humans.

Induced pluripotent stem (iPS) cells have recently been reported [Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131:861-872; Takahashi, K., and Yamanaka, S. 2006. Induction of pluripotent stem cells from mouse embryonic and adult fibroblast cultures by defined factors. Cell 126:663-676, Hanna, J., Wernig, M., Markoulaki, S., Sun, C. W., Meissner, A., Cassady, J. P., Beard, C., Brambrink, T., Wu, L. C., Townes, T. M., et al. 2007. Treatment of sickle cell anemia mouse model with iPS cells generated from autologous skin. Science 318:1920-1923; Maherali, N., Ahfeldt, T., Rigamonti, A., Utikal, J., Cowan, C., and Hochedlinger, K. 2008. A high-efficiency system for the generation and study of human induced pluripotent stem cells. Cell Stem Cell 3:340-345; Wernig, M., Meissner, A., Foreman, R., Brambrink, T., Ku, M., Hochedlinger, K., Bernstein, B. E., and Jaenisch, R. 2007. In vitro reprogramming of fibroblasts into a pluripotent ES-cell-like state. Nature 448:318-324]. By manipulating the expression of several transcription factors activated in ES cells (Oct4, Sox2, Nango, Lin28, c-Myc, and Klf4) through transduction with retrovirus expression vectors, adult fibroblasts have been reprogrammed to become cells that resemble ES cells with pluripotent characteristics.

### SUMMARY OF THE INVENTION

Methods are disclosed for making induced pluripotent stem (iPS) cells. The method comprises: isolating fetal somatic cells in utero from a fetus or from cord blood and reprogramming the fetal somatic cell to an iPS cell. The fetal cells are preferably isolated in utero by amniocentesis and from cord blood by use of appropriate antibodies to fetal cell surface antigens.

In some embodiments the fetal somatic cell contains a predetermined genetic defect. Such defects are generally in single genes (i.e. one and/or both alleles of a gene). Examples of diseases caused by such defercts include sickle cell disease, beta-thalassemia, cystic fibrosis, Tay-Sachs disease, adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), mucopolysaccharidosis and Lesch-Nyhan syndrome. Such genetic defects can be ascertained by analyzing fetal cells obtained by amniocentesis or CVS.

Reprogramming of the fetal somatic cell to an iPS cell is with a reprogramming vector comprising nucleic acid encoding one or more reprogramming factors. Such reprogramming vectors encode at least Oct4 and Sox2 but may also include one or more of Nango, Lin28, c-Myc, and Klf4. Retroviral vectors can be used, but is preferred that the vector is not a retroviral vector.

Preferably, the vector includes nucleic acid encoding one or more integration recognition sequences that are recognized by an integrase enzyme and which target the same or similar sequences in the genome of the fetal somatic cell for integration. Examples of integration sequences include attB and attP which can be used with the PhiC 31 integrase enzyme. The nucleic acid encoding the integration sequence is incorporated into the vector together with nucleic acid encoding the reprogramming factors and one or more selection markers. The vector is preferably a plasmid, such as that disclosed in FIG. 3. Preferably, a second vector containing nucleic acid encoding the integrase enzyme is co transfected into the fetal somatic cell to facilitate integration into the genome. The expression of the nucleic acid encoding the reprogramming genes is preferably transient and induced under tight expression control sequences to prevent undesirable continued gene expression.

When the fetal somatic cell contains a predetermined genetic defect it can be corrected in the somatic cell or the iPS cell. However, in the case of human fetal cells it is preferred that the defect be corrected in the iPS cell. The method of correcting of the predetermined genetic defect comprises transfecting the fetal somatic cell or iPS cell with a correction vector comprising nucleic acid encoding the correction of the predetermined genetic defect. It is preferred that the correction occur by homologous recombination between the genome of the cell and the correction vector. In some embodiments a vector comprising nucleic acid encoding a zinc finger nuclease is used with the correction vector to facilitate homologous recombination. The zinc finger nuclease is chosen so that it cleaves genomic DNA at or near the site of the genetic defect.

iPS cells can be converted to pluripotent stem cells. In the case of iPS cells where a genetic defect has been corrected the pluripotent stem cell can be use to treat the fetus or individual having the defect. Since the fetal somatic cells are derived from the same organism, graft rejection is minimalized.

Also disclosed are iPS cells and stem cells made according to the above methods. As compared to the use of retroviral vectors which randomly integrate into the genome, the iPS and stem cells are genotypically unique in that one or more reprogramming genes are located at one or more specific sites within the genome. IPS and stem cells having this genotype are obtained in a preferred embodiment by incorporating integrase recognition sequences in the reprogramming vector and transfecting the fetal somatic cells with the reprogramming vector and a second vector encoding an integrase enzyme. The fetal somatic cells can be normal or contain a genetic defect. Accordingly, the iPS cells and stem cells can be normal or contain a genetic defect. If the fetal somatic cell contains a genetic defect that is corrected in one or more alleles, the IPS and stem cells can be further characterized genotypically by the presence of a positive selection marker associated with the correction vector used to correct the defect by homologous recombination. In a preferred embodiment, a vector containing the HoxB4 gene, preferably using the cre-lox system, is used to increase the propagation of the hematopoietic stem cells or by stimulation of the endogenous Hox4B gene using double stranded activating RNA. Accordingly, when a vector encoding Hox4B is used such cells may be additionally characterized genotypically by the presence of the HoxB4 gene inserted into the genome by transfection. However, in this situation, it is preferred that the HoxB4 gene be removed via the cre-lox system after stem cell formation.

iPS and stem cells made according to the disclosed methods can be stored for later use as autologous grafts during the life of the individual that develops from the fetus or can be used to correct a genetic defect present in the fetus or individual. Such cells may also be used as allografts in conjunction with immunosuppressive therapy as may be appropriate

### BRIEF DESCRIPTION OF THE DRAWINGS

FIG. 1 depicts human ES cells compared to iPS cell colonies reprogrammed from cells from amniotic fluid (AF) and chorionic villus sampling (CVS). Insets showed alkaline phosphatase stain.

FIG. 2 depicts embroid-body mediated differentiation of iPS cells derived from amniotic fluid cells. A: Photomicrograph of differentiated iPS cells on day 16 (4×); B: immuno-stained with α-smooth muscle actin; C:βIII-Tubulin; and D: α-fetoprotein (20×).

FIG. 3 depicts the construction of the attB site-linked reprogramming vector. The transcription factor genes were placed under the control of the tight tetracycline response element (TRE-tight) and bracketed by the chicken insulator to control reprogramming factor gene expression and to shield it from leaky uninduced expression by the transactivator rtTA. O: Oct4, K: Klf4, S: Sox2, M: cMyc, g: GFP. Two constructs were made with the transcription factor gene order: OKSM and OSKM.

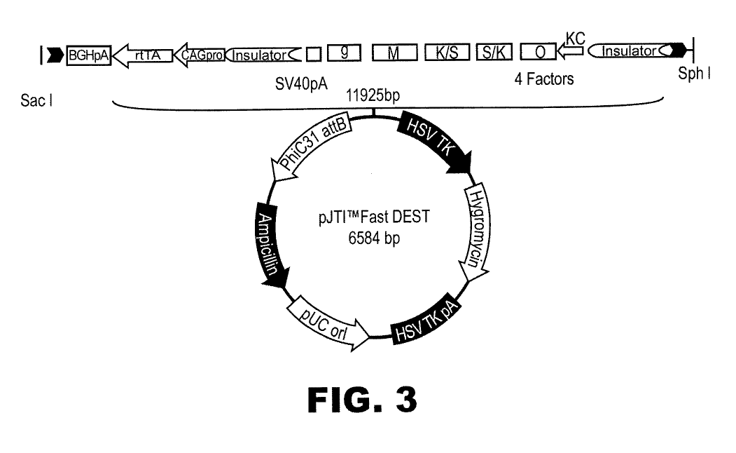

FIGS. 4A and 4B depicts the integration sites of the attB site-linked reprogramming vector in mouse and human chromosomes. The distances from the adjacent genes are shown. FIG. 4A shows the two different OSKM and OKSM constructs integrated into mouse chromosome 14 at the same site, illustrating the specificity of integration. FIG. 4B shows integration into the chromosomes of iPS cells prepared from human amniotic fluid.

FIG. 5 depicts two iPS cell colonies reprogrammed with 3 factors (3sy) or 4 factors (4sy) from the skin fibroblasts of a patient with homozygous β thalassemia (β-thal) compared to human embryonic stem cell colonies (hES). They were stained for alkaline phosphatase (AP, inset) and ES cell markers Nanog, SSEA3, SSEA4, Tra-1-60 and Tra-1-81.

FIG. 6A (SEQ ID Nos. 1 and 2) depicts the sequence analysis and FIG. 6B the karyotype of iPS cells reprogrammed from the β thalassemia patient's fibroblasts.

FIG. 7 shows photomicrographs of H&E stain of teratoma formed in NOD-SCID mice after injection of iPS cells from the β thalassemia patient showing, A) heterogeneous tissue under low power (4×), B) respiratory epithelium, C) bone and D) neural tissues (20×).

FIG. 8 depicts the differentiation of iPS cells into hematopoietic cells. Left panel: hematopoietic colonies (4×); middle: Giemsa stain showing hemoglobinization of some of the cells; and right: superimposition of DAPI and anti-HbF antibody stains (40×).

FIG. 9 depicts two nucleic acid constructs useful in correcting the point mutation in the beta-globin gene responsible for sickle cell disease.

### DETAILED DESCRIPTION OF THE INVENTION

Fetal somatic cells are used to make iPS cells and stem cells. The fetal cells are reprogrammed to form iPS cells and the iPS cells are thereafter transformed into stem cells. In some embodiments, a genetic defect is present in the fetal cell. This defect is corrected in the fetal cell, the iPS or the stem cell. The corrected stem cells differentiate to form the tissue affected by the disease, such as hematopoietic stem cells that differentiate into red blood cells. The corrected stem cells are returned to the patient to treat the genetic defect. In the case of hematopoietic stem cells, the preferred administration is injection into the vasculature. In utero treatment is preferably via injection into the vein of the umbilical cord. In the case of sickle cell disease and beta-thalassemia, the disease does not manifest until after birth when the child switches from fetal to adult hemoglobin. Treatment is therefore preferably post partum. However, in the case of other diseases, such as metabolic disorders that are a threat to fetal survival, it is preferred that the treatment be in utero or as soon after birth as possible.

Fetal somatic cells can be obtained in utero by fetoscopy, by amniocentesis or by chorionic villus sampling (CVS). Amniocentesis is the preferred method of obtaining fetal cells. The fetal cells are preferably obtained in the first or second trimester. This provides ample time to perform the disclosed methods by the time the child is born. In some diseases where the damage to the fetus may begin in utero, it is also possible to institute treatment in utero. Once the corrected iPS cells have been made, they can be stored or used to treat the genetic disease or other diseases that may develop over the course of that person's life.

Fetal somatic cells can also be obtained from cord blood (CB). Antibodies (typically immobilized on beads) against fetal surface antigens can be used for this purpose. Such antibodies include anti-CD34 and anti-CD133. Alternatively, fetal mononuclear cells can be obtained from CB by separation on Ficoll-Hypaque gradients. When CB derived fetal somatic cells are used, it is preferred that they be reprogrammed to iPS cells by use of a reprogramming vector containing integrase recognition sequences and a vector encoding an integrase that recognizes such integrase recognition sequences or an episomal vector encoding the reprogramming genes.

In one embodiment, the fetal somatic cells obtained for the purpose of prenatal diagnosis are the same cells used in the disclosed methods to provide iPS and the stem cells used for therapeutic treatment of a diagnosed genetic disease. A second procedure to obtain fetal somatic cells and its attendant risk to the pregnancy is thereby avoided.

Also disclosed are iPS cells and stem cells that contain one or more reprogramming genes that are located at one or more predetermined chromosomal positions. Such cells may also be characterized by the presence of a selection marker inserted into the genome by homologous recombination of a correction vector used to correct a genetic defect in the fetal somatic cell, the iPS cell or stem cell.

### I. Definitions

As used herein, the term “somatic cell” means a cell forming the body of an organism, as opposed to germline cells. In mammals, germline cells (also known as “gametes”) are the spermatozoa and ova which fuse during fertilization to produce a cell called a zygote, from which the mammalian embryo develops. Every other cell type in the mammalian body is a somatic cell. Internal organs, skin, bones, blood, and connective tissue are all made up of somatic cells.

As used herein, the term “fetal somatic cell” means somatic cells derived from a fetus. Generally, fetal cells are obtained by fetoscopy, chorionic villus sampling (CVS) or amniocentesis.

As used herein, the term “cell having a predetermined genetic defect refers” to a somatic cell which is know to harbor a genetic defect. A “fetal somatic cell having a predetermined genetic defect” refers to a fetal somatic cell which is known to harbor a known genetic defect.

As used herein, the term “genetic defect” means any modification within the genome of a somatic cell that causes a genetic disease. Examples include the substitution, insertion and/or deletion of one or more nucleotides within the genome of the somatic cell as well as the translocation and/or inversion of one or more nucleic acid segments.

As used herein, the term “genetic disease” means any disease state caused by a genetic defect. Examples include but are not limited to sickle cell disease, beta-thalassemia, cystic fibrosis, Tay-Sacs disease, adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), juvenile-onset, type 1 diabetes mellitus (JDM), Down syndrome (DS)/trisomy 21, and the carrier state of Lesch-Nyhan syndrome.

As used herein, the term “induced pluripotent stem cell”, or “iPS cell” means a pluripotent stem cell artificially derived from a non-pluripotent cell, such as an adult somatic cell or a fetal somatic cell as disclosed herein, by inducing the expression of certain genes disclosed in more detail herein.

As used herein, the term “reprogramming” refers to one or more steps needed to convert a somatic cell to an iPS cell. Reprogramming of fetal somatic cells to iPS cells is disclosed in more detail below.

As used herein, the term “stem cell” means a cell that has the ability to renew itself through mitotic cell division and differentiate into a diverse range of specialized cell types. There are two types of mammalian stem cells: embryonic stem cells (ES cells or ESC) that are derived from blastocysts, and adult stem cells or somatic stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

Stem cells are further characterized by their potency of differentiating into different cell types. Thus potency specifies the differentiation potential (the potential to differentiate into different cell types) of the stem cell. Stem cells are categorized into five groups according to their potency: (1) totipotent (or omnipotent) stem cells that can differentiate into embryonic and extra embryonic cell types; (2) pluripotent stem cells that are the descendants of totipotent cells and can differentiate into nearly all type of cells, i.e. cells derived from any of the three germ layers; (3) multipotent stem cells that can differentiate into a number of cells, but only those of a closely related family of cells (e.g. hematopoietic stem cells differentiate into red blood cells, white blood cells, platelets, etc.); (4) oligopotent stem cells that differentiate into only a few type of cells, such as lymphoid or myeloid stem cells; and (5) unipotent cells that can produce only one cell type, their own, but have the property of self-renewal which distinguishes them from non-stem cells (e.g. muscle stem cells).

As used herein, the term “transforming” refers to the steps required for the conversion of iPS cells to stem cells.

### II. Methods of Isolating Fetal Somatic Cells

Fetal somatic cells can be obtained by (1) withdrawal of amniotic fluid, a procedure known as amniocentesis (2) drawing fetal blood by fetoscopy and (3) chorionic villus sampling (CVS). Amniocentesis is the preferred procedure.

Alternatively, fetal somatic cells can be derived from cord blood.

### III. Methods of Reprogramming Fetal Cells to make iPS Cells

In the disclosed methods, iPS cells are made from fetal somatic cells. Such somatic cells may be normal or have a predetermined genetic defect. Alternatively, the fetal somatic cells can be fetal cells wherein a genetic defect has been corrected.

In one embodiment, the fetal somatic cells are reprogrammed by transient expression of one or more of the reprogramming genes such as the transcription factors Oct4, Sox2, Klf4, c-Myc, Nanog, and Lin28. In some embodiments the reprogramming is by transient expression of Oct4 and Sox with either Klf4 and c-Myc or Nanog and Lin 28. In other embodiments the reprogramming is by transient expression of c-Myc, Klf4, Sox2 and Oct4. In some embodiments the reprogramming is by transient expression of only Oct4 and Sox2. [Huangfu, D., Maehr, R., Guo, W., Eijkelenboom, A., Snitow, M., Chen, A. E., and Melton, D. A. 2008. Induction of pluripotent stem cells by defined factors is greatly improved by small-molecule compounds. Nat Biotechnol 26:795-797]. In still other embodiments, the fetal somatic cells are reprogrammed without the use expression sequences associated with SV 40 large T antigen.

When vectors are used that are integrated into the genome of the fetal cell, such vectors may optionally contain cre-lox sequences positioned so that the reprogramming genes can be removed after iPS or stem cell formation.

### Vector-Based iPS Reprogramming (Retroviral Vectors)

Retroviral integrating vectors encoding one or more reprogramming genes can be used but are not preferred because of the potential for insertional mutagenesis [Dave, 2004; Fischer, 2005].

To circumvent this obstacle, non-integrating adenovirus (Adeno) vectors [Stadtfeld, M., Nagaya, M., Utikal, J., Weir, G., and Hochedlinger, K. 2008. Induced pluripotent stem cells generated without viral integration. Science 322:945-949] or plasmids [Okita, K., Nakagawa, M., Hyenjong, H., Ichisaka, T., and Yamanaka, S. 2008. Generation of mouse induced pluripotent stem cells without viral vectors. Science 322:949-953] can be used to deliver the reprogramming genes to reprogram the fetal somatic cells into iPS cells. The genes can be introduced together with a single vector, or separately using two or more vectors. Adenoviral vectors are used that are similar to the vectors used successfully to reprogram mouse tail tip fibroblasts cells {Okita, K., Nakagawa, M., Hyenjong, H., Ichisaka, T., and Yamanaka, S. 2008. Generation of mouse induced pluripotent stem cells without viral vectors. Science 322:949-953; Stadtfeld, M., Nagaya, M., Utikal, J., Weir, G., and Hochedlinger, K. 2008. Induced pluripotent stem cells generated without viral integration. Science 322:945-949}. In those experiments a first generation adenovirus vector was used which retained some of the adenoviral genes and contained only one of the four transcription factor genes, cMyc, Sox2, klf4 and Oct4. This has the disadvantage of requiring simultaneous cell transduction by 4 individual vectors, possible cell toxicity by residual viral gene products, and a low efficiency of conversion into iPS cells. Alternatively, “gutless” or helper dependent adenoviral vectors which have the adenovirus ITR without any remaining viral genes can be used {Oka, K., and Chan, L. 2005. Construction and characterization of helper-dependent adenoviral vectors for sustained in vivo gene therapy. Methods Mol Med 108:329-350}. The genes encoding the 4 transcription factors are cloned into a single vector to create bi-genic expression cassettes linked with an IRES sequence or to create a tetra-genic expression cassette that utilizes the 2A ribosomal skipping system to express all four transcription factor genes under control of a single promoter {Szymczak, A. L., Workman, C. J., Wang, Y., Vignali, K. M., Dilioglou, S., Vanin, E. F., and Vignali, D. A. 2004. Correction of multi-gene deficiency in vivo using a single ‘self-cleaving’ 2A peptide-based retroviral vector. Nat Biotechnol 22:589-594}. For both vector systems, and because all 4 genes are linked within a single vector, transduced cells have high level, simultaneous co-expression of all the transcription factors. Since gene expression delivered by adenoviral vectors declines over about 48 hours, fibroblasts are transfected every other day for two weeks to maintain high level expression of the transcription factors {Stadtfeld, M., Nagaya, M., Utikal, J., Weir, G., and Hochedlinger, K. 2008. Induced pluripotent stem cells generated without viral integration. Science 322:945-949}. Because adenoviral vectors do not replicate and infrequently integrate, the vectors are lost through dilution during long term cell culture which allows inclusion of the cMyc gene which confers an increased efficiency of reprogramming.

Repeated transfection with plasmid vectors can reprogram fibroblasts into iPS cells {Okita, K., Nakagawa, M., Hyenjong, H., Ichisaka, T., and Yamanaka, S. 2008. Generation of mouse induced pluripotent stem cells without viral vectors. Science 322:949-953}. DNA plasmids containing one or more reprogramming genes are used to reprogram fetal somatic cells to iPS cells. For both these methods, the cells can be assayed for the infrequent integration of the viral or plasmid sequences by Southern blot and PCR.

### Vector-Based iPS Reprogramming (Integrase System)

A preferred method to reprogram fetal cells uses a vector containing one or more, preferably two or more, reprogramming genes in conjunction with integration sequence and an integrase enzyme. A preferred integrase system is that utilized by phage phi C31 although other integrase systems can be used. The phi C31 integrase system uses a vector encoding the phi C31 integrase and a reprogramming vector containing the reprogramming gene(s) and attP or attB sequences. See e.g. Chalberg, T. W., Portlock, J. L., Olivares, E. C., Thyagarajan, B., Kirby, P. J., Hillman, R. T., Hoelters, J., and Calos, M. P. 2006. Integration specificity of phage phiC31 integrase in the human genome. J Mol Biol 357:28-48; Groth, A. C., Olivares, E. C., Thyagarajan, B., and Calos, M. P. 2000. A phage integrase directs efficient site-specific integration in human cells. Proc Natl Acad Sci USA 97:5995-6000; Thyagarajan, B., Guimaraes, M. J., Groth, A. C., and Calos, M. P. 2000. Mammalian genomes contain active recombinase recognition sites. Gene 244:47-54; Thyagarajan, B., Olivares, E. C., Hollis, R. P., Ginsburg, D. S., and Calos, M. P. 2001. Site-specific genomic integration in mammalian cells mediated by phage phiC31 integrase. Mol Cell Biol 21:3926-3934; Thorpe H M, Wilson S E, Smith M C M. Control of directionality in the site-specific recombination system of of the Streptomyces phage PhiC31. Molecular Microbiology, 2000 38 232-241.

Other integrases from phage A118, Ui53, Bxb1, PhiFC1, PhiRV1 can be used. See e.g. Keravala A, Groth A C, Jarrahian S, Thyagarajan B, Hoyt J J, Kirby P J, Calos M P. A diversity of serine phage integrases mediate site-specific recombination in mammalian cells. Mol Genet Genomics. 2006 August; 276(2):135-46. Epub 2006 May 13.

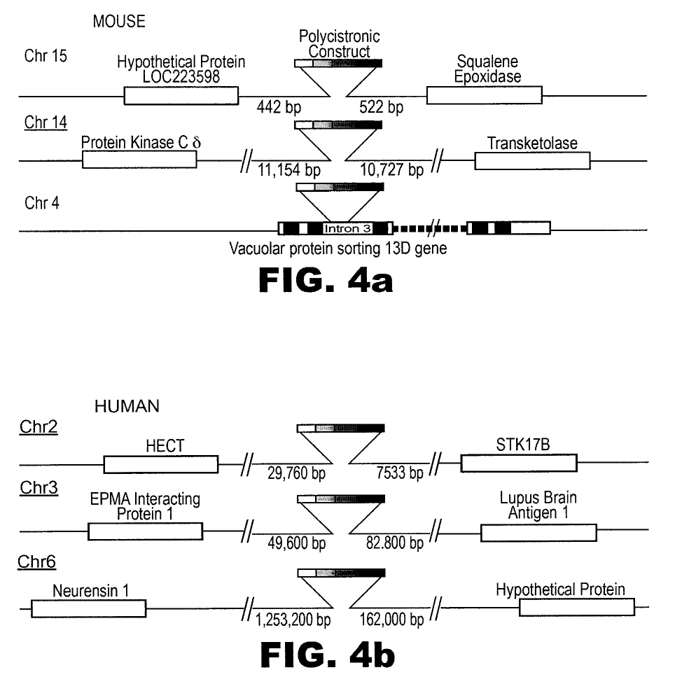

Examples 2 and 3 set forth the use of phi C31 phage integrase system to produce site specific genomic integration of a reprogramming vector into the genome of mouse embryonic and human fetal cells (See FIGS. 4A and 4B).

### Episomal Vectors

Episomal vectors can also be used to introduce reprogramming genes into fetal somatic cells to produce iPS cells. See e.g. Yu J, Hu K, Smuga-Otto K, Tian S, Stewart R, Slukvin I I, Thomson J A. Human induced pluripotent stem cells free of vector and transgene sequences. Science. 2009 May 8;324(5928):797-801. Epub 2009 Mar. 26.

### Characterization of iPS Cells

Potential iPS clones are evaluated for ES cell markers including telomerase, Nanog, Tra-1-60, Tra-1-81, SSEA-3, and SSEA-4 by immunostaining. Profiles of global gene expression and epigenetic markers are mapped by cDNA microarrays and ChIP-on-chip assays. The iPS cells are karyotyped and fingerprinted to confirm their fibroblast cell origin. To assess the differentiation potential of iPS cells, EB and teratoma formation assays are used {Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131:861-872}. The identities of differentiated cells is confirmed by immunostaining of tissue-specific markers such as βIII-tubulin (for ectoderm), α-SMA (for mesoderm), and AFP (for endoderm). To assay teratoma formation, cells are injected into immunocompromised (SCID) mice and the resulting teratomas are analyzed histologically for the presence of ectoderm-, mesoderm- and endoderm-derived tissues.

### IV. Transformation of iPS Cells to Stem Cells

A. Hematopoietic Stem Cells (HSCs)

There are several protocols for differentiating iPS cells into early hematopoietic lineages.

Method I: Co-Cultivation with Mouse OP9 Stromal Cells

iPS cells are grown in a methylcellulose-based medium to generate embroid bodies (EBs) {Chang, J. C., Ye, L., and Kan, Y. W. 2006. Correction of the sickle cell mutation in embryonic stem cells. Proc Natl Acad Sci USA 103:1036-1040}. After 9 days of culture, EBs are harvested, separated into single cells, plated on semiconfluent OP9 stromal cells {Nakano, 1994 #140} and grown in IMDM medium supplemented with, SCF, endothelia growth factor (VEGF), TPO, Flt-3 ligand {Kyba, 2002; Rideout, 2002}. In addition to the introduction of the HoxB4 gene flanked by loxP sites into the trypsinized EB cells {Rideout, 2002; Kyba, 2002} the EBs will be harvested and transferred to a flask with semiconfluent OP9 cells and fresh growth/differentiation medium for 14 days with or without the introduction of HoxB4 {Rideout, 2002}. HoxB4 transgenes will be flanked by loxP sites to allow removal of the gene after differentiation.

### Method 2

To induce formation of blast colony (BC)-forming cells, a two-step strategy is employed {Lu, S. J., Feng, Q., Caballero, S., Chen, Y., Moore, M. A., Grant, M. B., and Lanza, R. 2007. Generation of functional hemangioblasts from human embryonic stem cells. Nat Methods 4:501-509; Lu, S. J., Feng, Q., Park, J. S., Vida, L., Lee, B. S., Strausbauch, M., Wettstein, P. J., Honig, G. R., and Lanza, R. 2008. Biologic properties and enucleation of red blood cells from human embryonic stem cells. Blood 112:4475-4484}. Generation of early-stage EBs and induction of HSC precursor (mesoderm) formation is performed by plating iPS/hES cells on ultra-low attachment dishes and culturing for 2 days in medium containing BMP-4 and VEGF. The medium is then replaced and supplemented with a cocktail of early hematopoietic and endothelial growth factors (SCF, Tpo, FL, IL-3, G-CSF, IL-6; EPO, IGF-1, BMP-4, and VEGF) and triple protein transduction domain (tPTD)-HoxB4 fusion protein). After 3-5 days EBs are collected and dissociated. To expand the BL-CFC or HSCs, single cell suspensions are mixed with medium containing 1% methylcellulose in IMDM plus the above cocktail of growth factors plus tPTD-HoxB4 protein, and are cultured on ultra-low attachment dishes for another 4-6 days. The grape-like blast cell colonies from these cultures are visible by microscopy and can then be expanded further. For secondary colony growth, primary colonies are handpicked individually or pooled, dissociated into single cells and replated in the above cytokine cocktail with or without fusion protein.

### Method 3

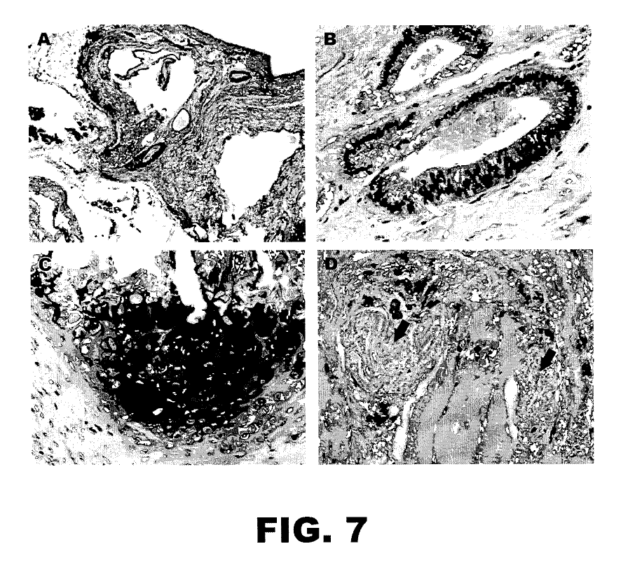

A preferred method utilizes two differentiation media. After washing, the iPS cells are resuspended in a first differentiation medium which is the basal medium, SFM supplemented with hBMP4, hVEGF, hSCF, hFlt3, hIL3, hIGF-II and TPO (Johansson, B. M., and Wiles, M. V. 1995. Evidence for involvement of activin A and bone morphogenetic protein 4 in mammalian mesoderm and hematopoietic development. Mol Cell Biol 15:141-151). Embryoid bodies (EBs) are made by plating these cells in a V-bottom 96-well plate and spun down in a centrifuge according to the method of Ng et al (Ng, E. S., Davis, R. P., Azzola, L., Stanley, E. G., and Elefanty, A. G. 2005. Forced aggregation of defined numbers of human embryonic stem cells into embryoid bodies fosters robust, reproducible hematopoietic differentiation. Blood 106:1601-1603; Ng, E. S., Davis, R. P., Hatzistavrou, T., Stanley, E. G., and Elefanty, A. G. 2008. Directed differentiation of human embryonic stem cells as spin embryoid bodies and a description of the hematopoietic blast colony forming assay. Curr Protoc Stem Cell Biol Chapter 1:Unit 1D 3). 5,000, 10,000 and 20,000 cells are plated in the wells to determine the optimum number for EB formation. EBs typically form when the cell number is greater than 10,000. After incubation at 37° C. and 5% CO2 for 10-14 days, EBs are then singly placed in a gelatinized tissue culture plate and allowed to differentiated further into hematopoietic lineage in a secondary differentiation medium which is SFM supplemented with hVEGF, hSCF, hFlt3, hIL6 and hEPO. Some fibroblast like cells may grow and spread out from the EB right away and some round, non-adherent cells may show up in some wells after 4-5 day incubation. Hemoglobinized colonies are typically evident with longer incubation. After 2 weeks, these non-adherent cells are removed, spun down on slides and stained with hemoglobin F antibody. The cells from the wells which contain hemoglobinized colonies show positive staining with antibody against hemoglobin F (See FIG. 8).

In a preferred embodiment, a vector containing the HoxB4 gene, preferably using the cre-lox system, is used to increase the propagation of the hematopoietic stem cells. After stem cell formation, the HoxB4 gene is removed.

Cells are analyzed throughout these culture processes. Immunological characterization by immunofluorescence microscopy is performed on cytospins of single cell suspensions from BL-CFU or hematopoietic colonies, and also on intact colonies following fixation and permeabilization, as appropriate, using standard methods.

B. Other Stem Cells

The following references disclose protocols that can be used for the formation of other types of stem cells that can be used to practice the invention.

Differentiation of iPS Cells into Neurons:

Dimos J T, Rodolfa K T, Niakan K K, Weisenthal L M, Mitsumoto H, Chung W, Croft G F, Saphier G, Leibel R, Goland R, Wichterle H, Henderson C E, Eggan K. Induced pluripotent stem cells generated from patients with ALS can be differentiated into motor neurons. Science. 2008 Aug. 29;321(5893):1218-21.

hES Cells into Liver Cells.

Cai J, Zhao Y, Liu Y, Ye F, Song Z, Qin H, Meng S, Chen Y, Zhou R, Song X, Guo Y, Ding M, Deng H. Directed differentiation of human embryonic stem cells into functional hepatic cells. Hepatology. 2007 May;45(Pawliuk, R., Westerman, K. A., Fabry, M. E., Payen, E., Tighe, R., Bouhassira, E. E., Acharya, S. A., Ellis, J., London, I. M., Eaves, C. J., et al. 2001. Correction of sickle cell disease in transgenic mouse models by gene therapy. Science 294:2368-2371):1229-39.

hES Cells into Beta Cells of Pancreas

Lees J G, Tuch B E. Conversion of embryonic stem cells into pancreatic beta-cell surrogates guided by ontogeny. Regen Med. 2006 May;1(Locatelli, F., Rocha, V., Reed, W., Bernaudin, F., Ertem, M., Grafakos, S., Brichard, B., Li, X., Nagler, A., Giorgiani, G., et al. 2003. Related umbilical cord blood transplantation in patients with thalassemia and sickle cell disease. Blood 101:2137-2143):327-36.

### IV. Correction of Genetic Defects

There are several approaches to correct genetic defects. The first is the use of classical vector-based gene targeting techniques utilizing positive-negative selection for homologous recombinant cells {Smithies, O., Gregg, R. G., Boggs, S. S., Koralewski, M. A., and Kucherlapati, R. S. 1985. Insertion of DNA sequences into the human chromosomal beta-globin locus by homologous recombination. Nature 317:230-234; Thomas, K. R., and Capecchi, M. R. 1987. Site-directed mutagenesis by gene targeting in mouse embryo-derived stem cells. Cell 51:503-512; Mansour, 1988}. Correction of the genetic defect is preferably made on iPS cells. Gene targeting in somatic and primary cell cultures is well established {Merrihew, 1995; Sargent, 1996; Brown, 1997; Bush, 1998}.

### Classical Vector-Based Homologous Recombination in Primary and iPS Cells

An example of a positive negative selection vector for targeted gene corrections of the sickle cell mutation is disclosed in Chang, 2006. This construct contains about 10 kb of homologous sequence to the normal human beta-globin gene, a hygromycin (hyg) positive-selectable marker and the negative-selectable herpes simplex TK gene. The gene correction construct can be used to correct the sickle cell defect in fetal somatic cells by transfected or in iPS cells by electroporation. The cells are selected with hyg and ganciclovir. Surviving colonies are isolated, expanded, and characterized by PCR and Southern hybridization to identify homologous recombinants. Since the hygromycin marker in this vector is flanked by loxP sequences, the marker is deleted in cells by transient transfection of a Cre recombinase expression vector to create cells that can be used to make iPS or stem cells for use in treating the disease.

### Characterization of Vector-Based Homologous Recombinants

Vector-based recombinant cell lines are analyzed by PCR to detect correction of the genetic defect {Maurisse, 2006}. Cells are either screened from clones or pools of cells grown in 96-well plates. When required, clonally unique homologous recombinant cell lines are isolated from screened pools. Normal and gene corrected genes in human primary, iPS, and HSC cells can be distinguished from defective alleles by PCR amplification of affected gene followed by restriction digestion of the PCR product. In the case of sickle cell disease, the restriction digest is with Ddel, Mstll, or Bsu36l {Goncz, 2006; Kan, 1977; Kan, 1992; Kan, 1978; Kan, 1978; Kan, 1976}. The presence of these cleavage sites indicates conversion from betaS- to betaA-globin. Random integration of vector sequences and wtSDFs can be assessed by inverse PCR (iPCR) {Ochman, 1988; Hartl, 1994; Hartl, 1996} or Southern blot hybridization of clonal isolates

### V. Applications

The disclosed methods provide cells that can be used to treat genetic disease. Examples of such diseases include inherited diseases of the hematopoietic system, the most common inherited diseases worldwide. Two notable members of this family of hemaglobinopathies are sickle cell disease (SCD) and beta-thalassemia (beta-thal) that result from mutations in the beta-globin and are common among the peoples of Africa, the Mediterranean, the Middle East, and Asia.

SCD and beta-thalassemia are genetic diseases that result in significant pain and organ damage due to occlusion of the blood vessels (SCD) or toxic iron levels from monthly blood transfusions (beta-thal). SCD is caused by a single base mutation in the beta-globin gene and results in deformed erythrocytes that clog blood vessels at low oxygen tension. Beta-thal is 1 of the 2 forms of thalassemia, alpha and beta; where alpha-thal fetuses die within the 3rd trimester or shortly after birth; and a beta-thal child is born healthy, but usually develops severe anemia within weeks or months and requires monthly blood transfusions to compensate for lack of adult hemoglobin. SCD is the result of an A>T transversion in exon 6 converting a glutamic acid into a valine in the beta-globin protein and results in defective adult hemoglobin. There are numerous other mutations in the beta-globin gene that give rise to beta-thal and are characterized by a lack of adult hemoglobin after birth.

Patients have limited therapeutic options and require lifelong care. Currently, blood diseases such as sickle cell anemia or thalassemia are largely treated symptomatically. The most effective treatments for SCD and beta-thal have been bone marrow or cord blood transplantation, which is dependent on the rare compatible donor and is complicated by graft-versus-host disease (GVHD). The disclosed methods provide an important alternative to conventional transplantation and overcome the need for finding a compatible donor. Conversion of a patient's somatic cells into HSCs provides a means for autologous transplantation of corrected cells and circumvents the issue of rejection. As iPS cells are derived from the patients themselves, they are therefore histocompatible and will not be rejected by the recipients. In addition, using fetal somatic cells from chorionic villus sampling (CVS) to generate iPS cells offers a means for therapeutic intervention soon after birth and a positive alternative to selective abortion.

Other diseases that are amenable to treatment using the disclosed methods include but are not limited to Tay-sacs disease, adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), juvenile-onset, type 1 diabetes mellitus (JDM), Down syndrome (DS)/trisomy 21, and the carrier state of Lesch-Nyhan syndrome. See e.g Park I H, Arora N, Huo H, Maherali N, Ahfeldt T, Shimamura A, Lensch M W, Cowan C, Hochedlinger K, Daley G Q. Disease-specific induced pluripotent stem cells. Cell. 2008 Sep. 5;134(Pawliuk, R., Westerman, K. A., Fabry, M. E., Payen, E., Tighe, R., Bouhassira, E. E., Acharya, S. A., Ellis, J., London, I. M., Eaves, C. J., et al. 2001. Correction of sickle cell disease in transgenic mouse models by gene therapy. Science 294:2368-2371):877-86.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Examples

Example 1

### iPS Cell Generation (Retroviral Infection)

To see if the CVS and amniotic fluid cells used for prenatal diagnosis can be utilized for potential therapy, we explored if these cells can be reprogrammed into iPS cells. We obtained anonymously cells that were used for routine prenatal diagnosis of chromosomal abnormalities.

Amniotic fluid cells were obtained by centrifugation from 10-15 ml amniotic fluid in a centrifuge tube at 1,000 rpm for 10 minutes, the supernatant removed and the cell resuspended in 2 ml of AminoMax (Invitrogen) transferred to two T-25 flasks, the volume made up to 5 ml each and cultured at 37° C. with 5% CO2.

Chorionic villi were placed in a petri dish containing 2.5 ml aspiration media (1% sodium heparin, 1% L-Glu, and 1% Pen-Strp in RPMI-1640), and thoroughly cleaned by removing the attached decidua and blood clots under a dissecting microscope. The medium was replaced with 2 ml of 10× Trypsin-EDTA (Invitrogen), and incubate at 37° C. for 25 minutes. The trypsin solution was replaced with 2 ml of Collagenase II (500 U/ml, Worthington Biochemical) and incubateed at 37° C. for 45 minutes. The cells in suspension were centrifuged and suspended in aminoMax and treated and cultured in T-25 flasks as for the amniotic fluid cells.

### Retroviral Vector Production and Infection of Cells

293FT cells for retroviral production were maintained in retroviral infection medium [Dulbecco's modified eagle medium (DMEM) containing 10% fetal bovine serum (Hyclone), 2 mM L-glutamine (Invitrogen) and 50 unit/ml penicillin and 50 mg/ml streptomycin]. They were co-transfected with pMXs retroviral vectors containing the human cDNAs for Oct3/4, Sox2, Klf4 and cMyc, VSV-G and Gag-Pol (pUVMC) (both from Addgene) with Fugene 6 (Roche). Forty-eight hours after transfection, the medium was collected, filtered and concentrated by ultra-centrifugation at 70,000×g for 1.5 hours at 4° C. The titer was adjusted to 1×10⁸ infectious units per ml in DMEM.

Skin fibroblast, CVS and amniotic fluid (AF) cells were seeded at 1×10⁵/well of a 6-well plate and 6 hours later the medium was replaced with the retroviral infection medium supplemented with 5 μg/ml of protamine sulfate, and containing the retroviral vectors Oct4, Sox2, Klf4 and cMyc for fibroblast cells and CVS cells, Oct4, Sox2 and Klf4 for fibroblast and AF cells, Oct4 and Sox2 for AF cells at the MOI of 10. Twenty-four hours later, the culture was replaced with the same medium without the viral vectors. Four to 5 days after infection, the cells were harvested by trypsinazation and replated at 1×10⁵ cells per 10 cm gelatin-coated dish on MEF feeder layer in retroviral infection medium. The medium was replaced the next day with human ES medium (DMEM/F12 medium supplemented with 20% Knockout serum replacement serum, 1 mM L-Glutamine, 100 μM nonessential amino acids, 100 μM β-mercaptoethanol, 50 U/ml penicillin and 50 mg/ml streptomycin, 4 ng/ml bFGF, all from Invitrogen) and supplemented with 2 mM valproic acid (Sigma). One week later, valproic acid was removed and cells were cultured with daily change of ES medium. Thirty to 35 days after transduction, colonies were picked up and transferred onto MEF feeder layer in 24-well plates. Five to 7 days later, colonies were expanded by mechanical dissociation or treated with collagenase and processed for analyses of marker gene expression and pluripotency.

The cells cultured from the CVS were infected with retroviral vectors carrying the 4 transcription factor genes. As iPS cells have been reported to be generated with fewer than 4 transcription factors, we infected the amniotic fluid cells with 2 vectors carrying the Oct3/4 and Sox2, or 3 vectors carrying the Oct3/4, Sox2 and Klf4 genes.

ES Cell Markers Identification

Alkaline phasphatase staining was performed with the kit from Millipore. For immunocytochemistry, iPS cells grown on feeder cells were fixed with 4% paraformaldehyde for 15 min. After washing with PBS, the cells were treated with PBS containing 5% normal goat or donkey serum (Vector), 1% BSA (Sigma), and 0.1% TritonX-100 for 45 min at room temperature. They were incubated with primary antibodies against SSEA3, TRA 1-81, TRA 1-60 (Millipore), Nanog (R&D system), or SSEA4 (Abcam) overnight at 4° C. followed the next day by 1 hour at room temperature with secondary antibodies, Alexa Fluor 555 conjugated goat anti-mouse IgM, Alexa Fluor 488-conjugated goat anti-mouse IgM, Alexa Fluor 546 conjugated goat anti-rabbit IgG, or Alexa Fluor 488-conjugated donkey anti-goat IgG, all from Invitrogen. The mounting solution (Vector) containing DAPI was used to counterstain the nuclei. Images were taken by using Pix Cell II (Arcturus) inverted fluorescence microscope and processed and analyzed using Adobe Photoshop software.

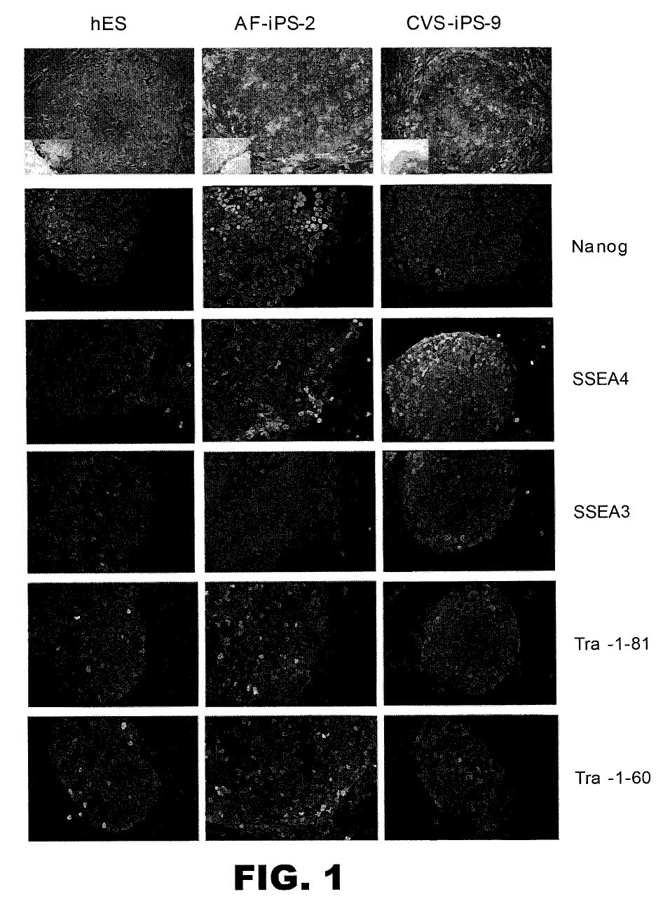

iPS cell colonies were successfully reprogrammed from all 3 samples of CVS and amniotic fluid cells and they expressed the pluripotency markers as in human ES cells (FIG. 1).

### Differentiation

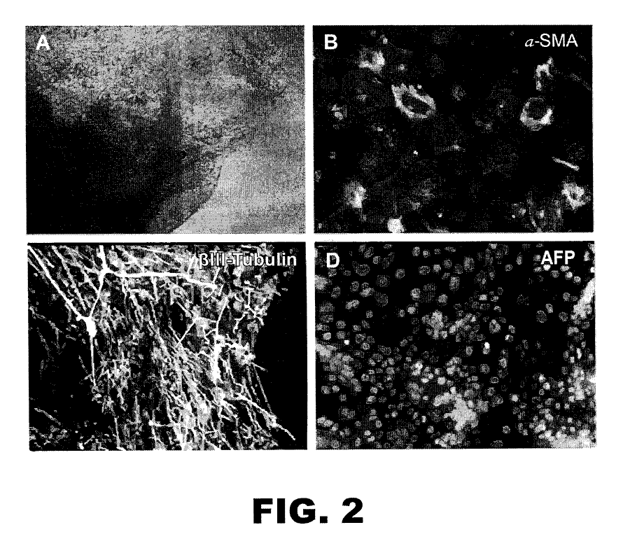

We tested the ability of the iPS cell prepared from prenatal diagnosis to differentiate into cells of the three germ layers in vitro. We used the EB mediated protocol to differentiate the iPS cells (Itskovitz-Eldor, J., Schuldiner, M., Karsenti, D., Eden, A., Yanuka, O., Amit, M., Soreq, H., and Benvenisty, N. 2000. Differentiation of human embryonic stem cells into embryoid bodies compromising the three embryonic germ layers. Mol Med 6:88-95). The iPS colonies were treated with collagenase, and cultured in human ES medium lacking bFGF. After 8 days in suspension culture, EBs were formed. The EBs were transferred to gelatin coated plates and further cultured for 8 days. We observed cells with diverse morphology (FIG. 2A), Immunocytochemistry analysis of these cells showed positive for α-smooth muscle actin (marker for mesoderm), βIII-tubulin (ectoderm),

and α-fetoprotein (endoderm), representative all 3 germ layers (FIG. 2B to D). These results indicate that the cells used for prenatal diagnosis have the potential to be reprogrammed to pluripotent stem cells that may be utilized for early treatment of genetic diseases.

### Example 2

### iPS Cell Generation (Phage Integrase System)

The following describes the use of a phage integrase system to reprogram mouse embryonic fibroblasts.

Construct of Phi C31 Integrase attB Site-Linked Reprogramming Vectors

In order to have high-level reprogramming factors expression, we took advantages of the tet-on (advanced) inducible gene expression system and placed rtTA-advanced under CAG promoter (gifted of Dr. Jun-ichi Miyazaki). cDNAs containing the open reading frames of human OCT4, Sox2, Klf4, cMyc and reporter GFP were combined into a single polycistronic construct connected by sequences encoding 2A peptides and placed under the tight tetracycline responsive element (TRE-tight) to control the programming factors and GFP genes. To place rtTA-advanced and the TRE-tight controlling the reprogramming factor genes into one vector as required by PhiC 31 integrase mediated integration system, we used the chicken beta-globin 5′ HS4 insulator element to flank the TRE-tight construct in order to prevent leaky expression of the reprogramming factors. We constructed two different order of reprogramming factors and named these as pJTI-Tet-on-Ins-OKSMG (Oct4, Klf4, Sox2, cMyc and GFP) (referred to as “OKSM”) and pJTI-Tet-on-Ins-OSKMG. (Oct4, Sox2, Klf4, cMyc and GFP) (referred to as “OSKM”). To verify inducible expression of each factor, we transfected 293T cells with the PhiC 31 integrase attB site-linked reprogramming vectors and analyzed protein expression by western blots and GFP expression by fluorescent microscope. The factors expression was doxycyline dependent.

Generation of Site Specific Integration iPS Cells Using the attB Site-Based Reprogramming Vectors with Phic 31 Integrase Vector

We next examined whether site specific integration could be mediated with PhiC 31 integrase. Mouse embryonic fibroblasts (MEF) were transfected by electroporation with 0.5 μg of the attB site-linked reprogramming vectors along with 10 μg of a plasmid that expresses PhiC31 integrase. For comparison, we also transfected into MEF cells the same amount the attB site-linked reprogramming vectors along with an irrelevant plasmid to bring the total DNA amount to 10.5 μg. Immediately after transfection, the cells were seeded on the gelatin-coated plates with or without feeder. The cells were cultured in mouse ES medium with 2 μg/ml of doxycycline 24 hours after transfection and continued 2 ug/ml of doxycycline for 10 days, then changed to 0.4 μg/ml of doxycycline and treated for another 4 days. On day 20, 29 colonies appeared on the plates with the reprogramming vector co-transfected with Phi C31 integrase. In contrast, only one colony appeared when the reprogramming vector was transfected without integrase. We noticed that culturing transfectants with the feeder cell could get 3 times more colonies than culturing without feeder cells. More than half of colonies could turn into stable iPS cells with criteria typical of embryonic stem cells such as morphology, pluripotency-marker expression, and teratoma formation. They also showed de-methylation in Oct4 and Nanog promoters. Southern blot analysis revealed that all the clones we isolated had only one integration site.

### Determination of the Integration Sites

We determined the sites of integration in 8 lines of iPS cells by plasmid rescue. 4 lines (MEFintOS-1, MEFintOK-2, MEF intOK-7, MEFintOk-8), the plasmid was successfully rescued and showed site specific integration, 2 lines (MEFint OK-4, MEF intOK-5) showed non-specific integration, the remaining 2 lines (MEFintOS-4 and MEF intOK-3) could not be rescued for technical reasons. The integration sites of the 4 lines that showed specific integrations are shown in the FIG. 4A. Notably, two independent lines with different constructs showed the same site of integration.

### Example 3

iPS Cell Generation from Human Amniotic Fluid Cells (Phage Integrase System)

The OKSM construct from Example 2 was used to make iPS cells out of amniotic fluid cells using essentially the same method as described in Example 2. However, compared to mouse (MEF) cells, human cells need to be cultured and stimulated with doxycycline for a much longer time period. No matter which method is used to make iPS cells, it takes longer to get human iPS cells as compared to mouse iPS cells.

### Generation of Human Amniotic Fluid iPS Cells Using Site Specific Integration

To determine if the same system could induce human somatic cells reprogram, we used cells from human amniotic fluid.

Amniotic fluid cells were obtained anonymously from clinical samples. Amniotic fluid cells were obtained by centrifugation from 10-15 ml amniotic fluid in a centrifuge tube at 1,000 rpm for 10 minutes, the supernatant removed and the cells resuspended in 2 ml of AminoMax (Invitrogen) transferred to two T-25 flasks, the volume made up to 5 ml each and cultured at 37° C. with 5% CO2.

Using nucleofactor, the transfection efficiency reached 70-80%. Since construct OKSM gave more colonies than construct OSKM in mouse experiments, we decided to use construct OKSM for the human experiment. We transfected 1×10⁶ cells with reprogramming factors construct OKSM along with Phic 31 integrase (OKSM/Phic 31) or reprogramming factors construct OKSM only. The reprogramming factors expression was induced by 1.5 mg/ml-2 mg/ml doxycycline 24 hours after transfection and cultured in 15% FBS, DMEM medium for 4 days. One the day 5, the medium was changed to human ES medium with same amount of doxycycline. The colonies started to appear on day 8 after transfection. We observed 8 iPS-like colonies from OKSM with Phic31, and 7 iPS-like colonies from construct OKSM only. We picked up 3 colonies on day 18 from the OKSM/Phi C31 plates and another 3 clones from OKSM only plates and stopped the use of doxycycline. These six clones rapidly flattened and returned to a fibroblast-like state after doxycycline withdrawal. Two weeks later, these clones were treated with doxycycline at a different dose. Two stable iPS lines from OKSM/phic31: AFint OK-3 using 5 mg doxycycline/ml for 8 days, and AF int OK-5 using 3 ug doxycycline/ml for 8 days, and 1 line from OKSM, AF-OK-1 using 0.5 mg doxycycline/ml for 10 days, total in about two to three weeks. We kept the original plates and continued the treatment with lower dose (0.5 ug/ml) of Doxycycline from day 20. One month later, MEF condition medium was used instead of Human ES medium. Four colonies were picked from the original OKSM/Phic31 plate on day 35 to day 45 and generated another 3 stable iPS lines (AFintOk-2, AFint Ok-7, AFintOK-12). These 6 iPS lines were maintained in Human ES medium without Doxycycline. All lines displayed human ES cell morphology and were positive for alkaline phosphatase, expression of endogenous pluripotent markers Nanog, SSEA3, SSEA4, Tra-1-60, Tra-1-81 were confirmed in all cell lines. One of the cell line successfully generated teratoma in 4 weeks after intra-muscular injection into NOD-SCID mouse. The teratoma showed various tissues and confirmed they contained three germ layers. We also analyzed the karyotype of AF intOK-2 cell line, it showed the normal 46 XX chromosome which is different with our previous generated AF iPS cell line (karyotype: 46 XY) using the retrovirus infection method.

The integration sites of the reprogramming vector in human fetal amniotic cells are shown in FIG. 4B.

### Example 4

### Reprogramming β-Thalassemia Fibroblasts

Fibroblasts cultures from skin biopsies were provided anonymously from a patient with homozygous beta⁰ thalassemia due to a 4-bp deletion frameshift mutation (—CTTT). Skin fibroblast cells were maintained in Dulbecco's modified eagle medium (DMEM, Invitrogen) containing 10% fetal bovine serum (FBS, Hyclone), 2 mM L-glutamine (Invitrogen) and 50 unit/ml penicillin/50 mg/ml streptomycin.

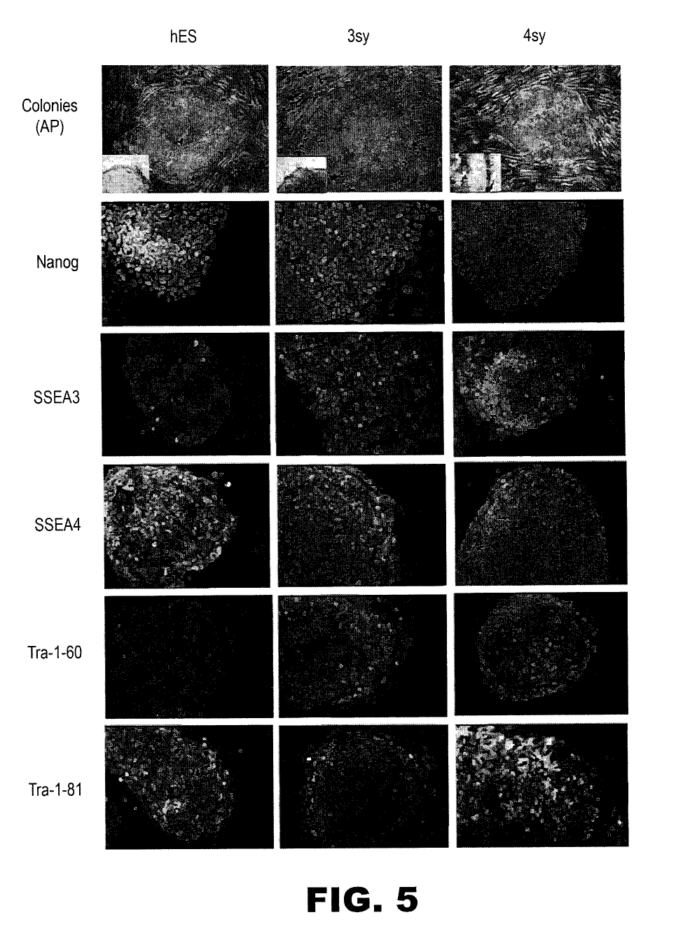

The codon 41/42 4-basepair (CTTT) deletion causes a frameshift and no β-globin chain is made. Cells from CVS and amniocentesis were obtained anonymously from the clinical laboratories at UCSF. Two types of infection were done in the generation of iPS cells from the skin fibroblasts by retrovirus vectors. In one, the skin fibroblasts were infected with retrovirus vectors containing the four transcription factor genes (Oct4, Sox2, Klf4 and cMyc), and in the other with three genes omitting cMyc With the addition of valproic acid in the culture medium, about 450 and 50 tightly packed colonies similar to human ES cell colonies began to appear at 2 to 3 weeks (Huangfu, D., Maehr, R., Guo, W., Eijkelenboom, A., Snitow, M., Chen, A. E., and Melton, D. A. 2008. Induction of pluripotent stem cells by defined factors is greatly improved by small-molecule compounds. Nat Biotechnol 26:795-797) after infection of 1×10⁵ cells with 4 and 3 factor genes respectively. Forty of 4 factors induced colonies (4sy series) and 30 of 3 factors induced colonies (3sy series) were picked at 30 to 35 days after infection. Thirty-five of 4 factors infected colonies and 28 of the 3 factors infected colonies could be expanded and showed the same morphology as human embryonic stem (ES) cells. They expressed the pluripotency markers, such as Nanog, SSEA3, SSEA4, Tra-1-60, Tra-1-81 as well as alkaline phosphatase, similar to human ES cells (FIG. 5). Western blot analysis of these colonies revealed similar level of Nanog expression compared to human ES cells (data not show). These cells could be maintained in an undifferentiated state on Matrigel-coated plates in MEF-conditioned human ES cell medium. Some of the colonies have been cultured for more than 4 to 7 months in our laboratory.

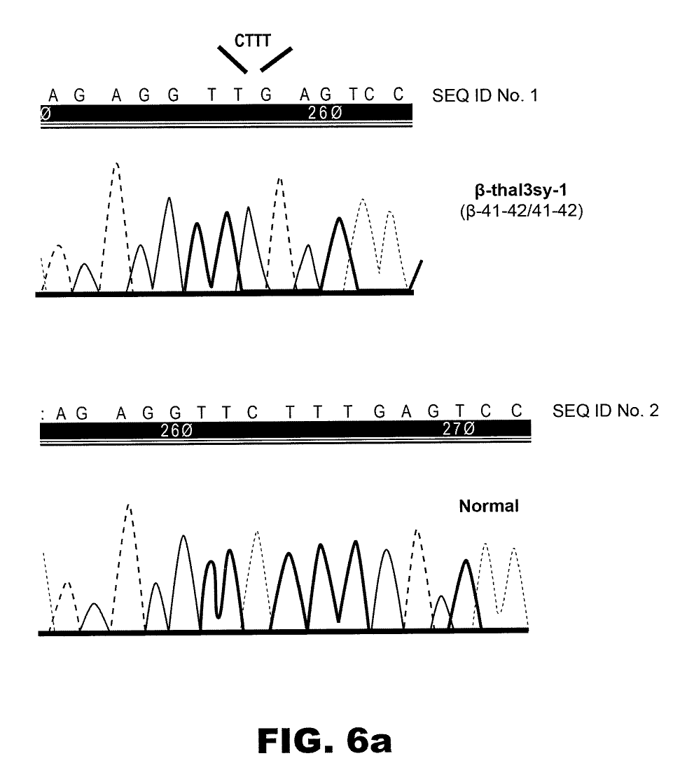

To confirm that the generated iPS cell lines were derived from the β-thalassemia patient's skin fibroblasts, genomic DNA sequencing analyses were performed in 5 individual iPS cell lines. Homozygous codon 41/42 4-bp (CTTT) deletion was verified in all 5, identical to the sequence of the skin fibroblasts (FIG. 6A). Karyotypes of 5 individual iPS lines after 5 passages were normal and one, cultured for 15 additional passages was also found to maintain the normal karyotype (FIG. 6B).

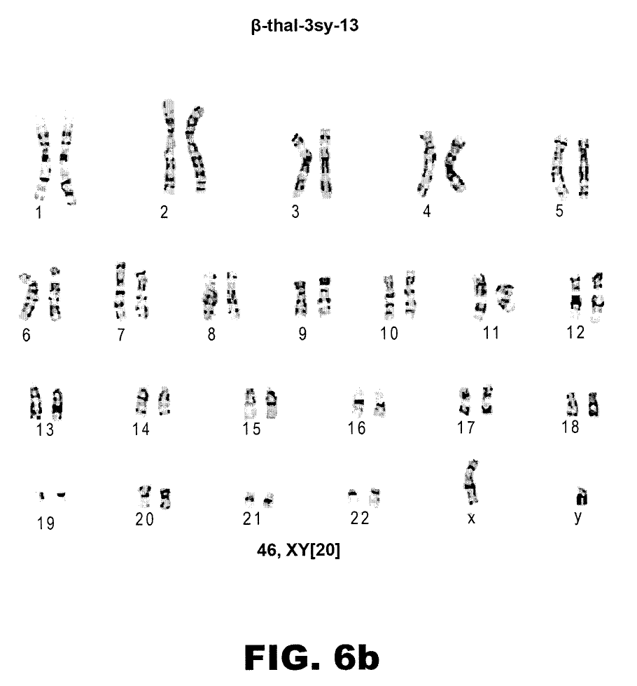

To examine the pluripotency of the generated iPS cells in vivo, we injected 10⁶ iPS cells intramuscularly into the hind-leg of immunodeficient (NOD-SCID) mice. Seven weeks after injection, tumor formation was observed. Histological analysis of the tumors revealed that they contained tissues representative of all three germ layers including ciliary respiratory and gut-like epithelia (endoderm), bone and muscle (mesoderm), nerve and sebaceous glands (ectoderm) (FIG. 7).

### Example 5

Transformation from iPS Cells to Hematopoietic Cells

We investigated the ability of the iPS cells from example 4 to differentiate into hematopoietic cells. Single cells were made from the iPS colonies with the enzyme Accutase. After washing, the cells were resuspended in the first differentiation medium which is the basal medium, SFM supplemented with hBMP4, hVEGF, hSCF, hFlt3, hIL3, hIGF-II and TPO (Johansson, B. M., and Wiles, M. V. 1995. Evidence for involvement of activin A and bone morphogenetic protein 4 in mammalian mesoderm and hematopoietic development. Mol Cell Biol 15:141-151). Embryoid bodies (EBs) were made by plating these cells in a V-bottom 96-well plate and spun down in a centrifuge according to the method of Ng et al (Ng, E. S., Davis, R. P., Azzola, L., Stanley, E. G., and Elefanty, A. G. 2005. Forced aggregation of defined numbers of human embryonic stem cells into embryoid bodies fosters robust, reproducible hematopoietic differentiation. Blood 106:1601-1603; Ng, E. S., Davis, R. P., Hatzistavrou, T., Stanley, E. G., and Elefanty, A. G. 2008. Directed differentiation of human embryonic stem cells as spin embryoid bodies and a description of the hematopoietic blast colony forming assay. Curr Protoc Stem Cell Biol Chapter 1:Unit 1D 3). Initially, we optimized the formation of EB by plating 5,000, 10,000 and 20,000 cells in the wells. EBs were formed in the well where the cell number is greater than 10,000. After incubation at 37° C. and 5% CO2 for 10-14 days, EBs were then singly placed in a gelatinized tissue culture plate and allowed to differentiated further into hematopoietic lineage in the secondary differentiation medium which is SFM supplemented with hVEGF, hSCF, hFlt3, hIL6 and hEPO. Some fibroblast like cells grew and spread out from EB right away and some round, non-adherent cells showed up in some wells after 4-5 day incubation and hemoglobinized colonies were evident with longer incubation. After 2 weeks, these non-adherent cells were removed, spun down on the slides and stained with hemoglobin F antibody. The cells from the wells which contain hemoglobinized colonies showed positive staining with antibody against hemoglobin F (FIG. 8).

### Example 6

### Treatment of Sickle Cell Anemia

Fetal cells obtained from amniotic fluid or from CVS are analyzed to determine if the cells contain the normal beta globin gene and/or the beta globin sickle cell gene.

Fetal cells homozygous for the sickle cell gene are converted into iPS cells using the protocol described in Example 2.

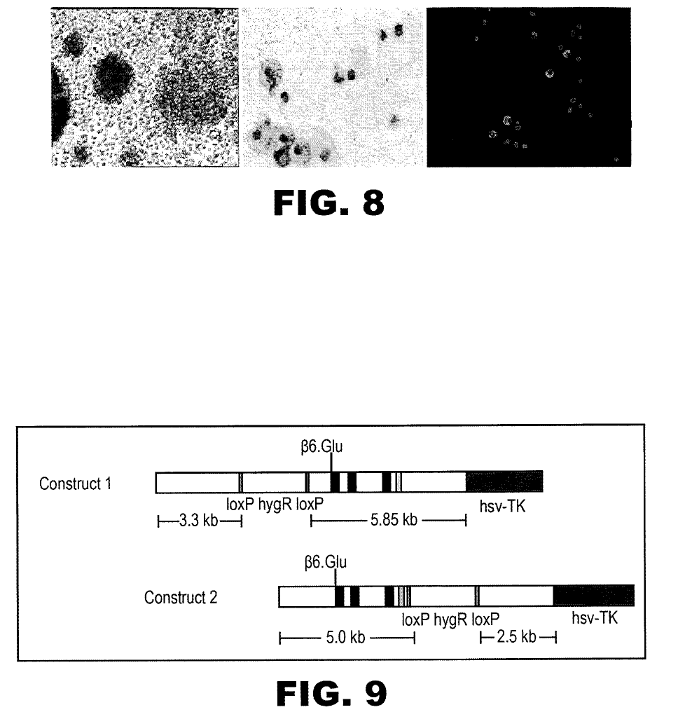

The sickle cell genetic defect is then corrected in the iPS cells by converting at least one of the sickle cell alleles to the normal beta globin gene using homologous recombination vectors such as those disclosed in FIG. 9 and Chang et al. PNAS 103, 1036-1040 (2006).

The corrected iPS cells are differentiated into hematopoietic stem cells using the protocol disclosed in example 5. A vector containing the HoxB4 gene, preferably using the cre-lox system, is used to increase the propagation of the hematopoietic stem cells. After stem cell formation, the HoxB4 gene is removed.

The stem cells are then transplanted in utero into the fetus or after birth using standard transplantation protocols.

### BIBLIOGRAPHY

1. Takahashi, K., Okita, K., Nakagawa, M. & Yamanaka, S. (2007) Nat Protoc 2, 3081-9.
2. Takahashi, K., Tanabe, K., Ohnuki, M., Narita, M., Ichisaka, T., Tomoda, K. & Yamanaka, S. (2007) Cell 131, 861-72.
3. Takahashi, K. & Yamanaka, S. (2006) Cell 126, 663-76.
4. Yamanaka, S. (2007). Cell Stem Cell 1, 39-49.
5. Dave, U. P., Jenkins, N. A. & Copeland, N. G. (2004) Science 303, 333.
6. Fischer, A. & Cavazzana-Calvo, M. (2005) PLoS Med 2, e10.
7. Stadtfeld, M., Nagaya, M., Utikal, J., Weir, G. & Hochedlinger, K. (2008) Science.
8. Okita, K., Nakagawa, M., Hyenjong, H., Ichisaka, T. & Yamanaka, S. (2008) Science.
9. Huangfu, D., Osafune, K., Maehr, R., Guo, W., Eijkelenboom, A., Chen, S., Muhlestein, W. & Melton, D. A. (2008) Nat Biotechnol.
10. Chen, Z., Place, R., Jia, Z., Pookot, D., Dahiya, R. & Li, L. (2008) Mol Cancer Ther 7, 698-703.
11. Li, L. C., Okino, S. T., Zhao, H., Pookot, D., Place, R. F., Urakami, S., Enokida, H. & Dahiya, R. (2006) Proc Natl Acad Sci USA 103, 17337-42.
12. Place, R., Li, L., Pookot, D., Noonan, E. & Dahiya, R. (2008) Proc Natl Acad Sci USA 105,1608-13.
13. Chang, J. C., Ye, L. & Kan, Y. W. (2006) Proc Natl Acad Sci USA 103, 1036-40.
14. Hanna, J., Wernig, M., Markoulaki, S., Sun, C. W., Meissner, A., Cassady, J. P., Beard, C., Brambrink, T., Wu, L. C., Townes, T. M. & Jaenisch, R. (2007) Science 318, 1920-3.
15. Smithies, O., Gregg, R., Boggs, S., Koralewski, M. & Kucherlapati, R. (1985) Nature 317,230-4.
16. Thomas, K. R. & Capecchi, M. R. (1987) Cell 51, 503-12.
17. Goncz, K. K., Prokopishyn, N. L., Abdolmohammadi, A., Bedayat, B., Maurisse, R., Davis, B. R. & Gruenert, D. C. (2006) Oligonucleotides 16, 213-24.
18. Goncz, K., Prokopishyn, N., Chow, B., Davis, B. & Gruenert, D. (2002) Gene Therapy 9, 691-4.
19. Gruenert, D. C., Bruscia, E., Novelli, G., Colosimo, A., Dallapiccola, B., Sangiuolo, F. & Goncz, K. K. (2003) J Clin Invest 112, 637-41.
20. Sangiuolo, F., Filareto, A., Spitalieri, P., Scaldaferri, M. L., Mango, R., Bruscia, E., Citro, G., Brunetti, E., De Felici, M. & Novelli, G. (2005) Hum Gene Ther 16, 869-80.
21. Sangiuolo, F., Scaldaferri, M., Filareto, A., Spitalieri, P., Guerra, L., Favia, M., Caroppo, R., Mango, R., Bruscia, E., Gruenert, D., Casavola, V., De Felici, M. & Novelli, G. (2008) Front Biosci 13,2989-99.
22. Carroll, D. (2008) Gene Ther 15, 1463-8.
23. Carroll, D., Morton, J. J., Beumer, K. J. & Segal, D. J. (2006) Nat Protoc 1, 1329-41.
24. Porteus, M. (2007) Biotechnol Genet Eng Rev 24, 195-212.
25. Porteus, M. (2008) Methods Mol Biol 435, 47-61.
26. Chang, K. H., Nelson, A. M., Cao, H., Wang, L., Nakamoto, B., Ware, C. B. & Papayannopoulou, T. (2006) Blood 108, 1515-23.
27. Lu, S. J., Feng, Q., Caballero, S., Chen, Y., Moore, M. A., Grant, M. B. & Lanza, R. (2007) Nat Methods 4, 501-9.
28. Lu, S., Feng, Q., Ivanova, Y., Luo, C., Li, T., Li, F., Honig, G. & Lanza, R. (2007) Stem Cells Dev 16, 547-59.
29. Lu, S., Luo, C., Holton, K., Feng, Q., Ivanova, Y. & Lanza, R. (2008) Regen Med 3, 693-704.
30. Stadtfeld, M., Brennand, K. & Hochedlinger, K. (2008) Curr Biol 18, 890-4.
31. Oka, K. & Chan, L. (2005) Methods Mol Med 108, 329-50.
32. Szymczak, A. L., Workman, C. J., Wang, Y., Vignali, K. M., Dilioglou, S., Vanin, E. F. & Vignali, D. A. (2004) Nat Biotechnol 22, 589-94.
33. Li, L. C. (2008) RNA Biol 5, 61-4.
34. Mansour, S. L., Thomas, K. R. & Capecchi, M. R. (1988) Nature 336, 348-52.
35. Kunzelmann, K., Legendre, J. Y., Knoell, D. L., Escobar, L. C., Xu, Z. & Gruenert, D. C. (1996) Gene Ther 3, 859-67.
36. Colosimo, A., Guida, V., Antonucci, I., Bonfini, T., Stuppia, L. & Dallapiccola, B. (2007) Haematologica 92, 129-30.
37. Merrihew, R. V., Sargent, R. G. & Wilson, J. H. (1995) Somat Cell Mol Genet 21, 299-307.
38. Sargent, R. G., Merrihew, R. V., Nairn, R., Adair, G., Meuth, M. & Wilson, J. H. (1996) Nucleic Acids Res 24, 746-53.
39. Brown, J. P., Wei, W. & Sedivy, J. M. (1997) Science 277, 831-4.
40. Bush, A., Mateyak, M., Dugan, K., Obaya, A., Adachi, S., Sedivy, J. & Cole, M. (1998) Genes Dev 12, 3797-802.
41. Davis, B. R., Yannariello-Brown, J., Prokopishyn, N. L., Luo, Z., Smith, M. R., Wang, J., Carsrud, N. D. & Brown, D. B. (2000) Blood 95, 437-44.
42. Graessmann, M. & Graessmann, A. (1983) Methods Enzymol 101, 482-92.
43. Hamm, A., Krott, N., Breibach, I., Blindt, R. & Bosserhoff, A. K. (2002) Tissue Eng 8, 235-45.
44. Porteus, M. H. (2006) Mol Ther 13, 438-46.
45. Porteus, M. H. & Baltimore, D. (2003) Science 300, 763.
46. Sargent, R. G., Brenneman, M. A. & Wilson, J. H. (1997) Mol Cell Biol 17, 267-77.
47. Urnov, F. D., Miller, J. C., Lee, Y. L., Beausejour, C. M., Rock, J. M., Augustus, S., Jamieson, A. C., Porteus, M. H., Gregory, P. D. & Holmes, M. C. (2005) Nature 435, 646-51.
48. Maurisse, R., Fichou, Y., De Semir, D., Cheung, J., Ferec, C. & Gruenert, D. C. (2006) Oligonucleotides 16, 375-86.
49. Kan, Y. W., Golbus, M. S. & Trecartin, R. (1976) N Engl J Med 294, 1039-40.
50. Ochman, H., Gerber, A. S. & Hartl, D. L. (1988) Genetics 120, 621-3.
51. Hartl, D. L. & Ochman, H. (1994) Methods Mol Biol 31, 187-96.
52. Hartl, D. L. & Ochman, H. (1996) Methods Mol Biol 58, 293-301.
53. Sedelnikova, O., Rogakou, E., Panyutin, I. & Bonner, W. (2002) Radiat Res 158, 486-92.
54. Schmitt, R. M., Bruyns, E. & Snodgrass, H. R. (1991) Genes Dev 5, 728-40.
55. Wiles, M. V. & Keller, G. (1991) Development 111, 259-67.
56. Nakano, T., Kodama, H. & Honjo, T. (1994) Science 265, 1098-101.
57. Kyba, M., Perlingeiro, R. C. & Daley, G. Q. (2002) Cell 109, 29-37.
58. Rideout, W., Hochedlinger, K., Kyba, M., Daley, G. & Jaenisch, R. (2002) Cell 109, 17-27.
59. Lu, S. J., Feng, Q., Park, J. S., Vida, L., Lee, B. S., Strausbauch, M., Wettstein, P. J., Honig, G. R. & Lanza, R. (2008) Blood.
60. Papayannopoulou, T., Brice, M. & Stamatoyannopoulos, G. (1986) Cell 46, 469-76.
61. Papayannopoulou, T., Nakamoto, B., Agostinelli, F., Manna, M., Lucarelli, G. & Stamatoyannopoulos, G. (1986) Blood 67, 99-104.
62. Papayannopoulou, T. H., McGuire, T. C., Lim, G., Garzel, E., Nute, P. E. & Stamatoyannopoulos, G. (1976) Br J Haematol 34, 25-31.
63. Stamatoyannopoulos, G., Farquhar, M., Lindsley, D., Brice, M., Papayannopoulou, T. & Nute, P. E. (1983) Blood 61, 530-9.
64. Dimos, J., Rodolfa, K., Niakan, K., Weisenthal, L., Mitsumoto, H., Chung, W., Croft, G., Saphier, G., Leibel, R., Goland, R., Wichterle, H., Henderson, C. & Eggan, K. (2008) Science 321, 1218-21.
65. Ishikawa, F., Yasukawa, M., Lyons, B., Yoshida, S., Miyamoto, T., Yoshimoto, G., Watanabe, T., Akashi, K., Shultz, L. D. & Harada, M. (2005) Blood 106, 1565-73.
66. Shultz, L., Lyons, B., Burzenski, L., Gott, B., Chen, X., Chaleff, S., Kotb, M., Gillies, S., King, M., Mangada, J., Greiner, D. & Handgretinger, R. (2005) J Immunol 174, 6477-89.
67. Baersch, G., Mollers, T., Hotte, A., Dockhorn-Dworniczak, B., Rube, C., Ritter, J., Jurgens, H. & Vormoor, J. (1997) Klin Padiatr 209, 178-85.
68. Greiner, D. L., Hesselton, R. A. & Shultz, L. D. (1998) Stem Cells 16, 166-77.
69. Shultz, L. D., Banuelos, S., Lyons, B., Samuels, R., Burzenski, L., Gott, B., Lang, P., Leif, J., Appel, M., Rossini, A. & Greiner, D. L. (2003) Transplantation 76, 1036-42.
70. Shultz, L., Lang, P., Christianson, S., Gott, B., Lyons, B., Umeda, S., Leiter, E., Hesselton, R., Wagar, E., Leif, J., Kollet, O., Lapidot, T. & Greiner, D. (2000) J Immunol 164, 2496-507.
71. Aiuti, A., Slavin, S., Aker, M., Ficara, F., Deola, S., Mortellaro, A., Morecki, S., Andolfi, G., Tabucchi, A., Carlucci, F., Marinello, E., Cattaneo, F., Vai, S., Servida, P., Miniero, R., Roncarolo, M. G. & Bordignon, C. (2002) Science 296, 2410-3.
72. Shaw, P. J., Nath, C., Berry, A. & Earl, J. W. (2004) Bone Marrow Transplant 34, 197-205.
73. Dravid, G., Hammond, H. & Cheng, L. (2006) Methods Mol Biol 331, 91-104.
74. Mali, P., Ye, Z., Hommond, H., Yu, X., Lin, J., Chen, G., Zou, J. & Cheng, L. (2008) Stem Cells 26, 1998-2005.
75. Oka, K. & Chan, L. (2005) Curr Protoc Mol Biol Chapter 16, Unit 16 24.
76. Chang, J., Lu, R., Lin, C., Xu, S., Kan, Y., Porcu, S., Carlson, E., Kitamura, M., Yang, S., Flebbe-Rehwaldt, L. & Gaensler, K. (1998) Proc Natl Acad Sci USA 95, 14886-90.
77. Gaensler, K., Poreu, S., Kitamura, M., Lin, C. & Kan, Y. (1995) Blood 86, 586a (Abst. 2332).
78. Chang, J., Lu, R., Xu, S., Meneses, J., Chan, K., Pedersen, R. & Kan, Y. (1996) Blood 88, 1846-51.

### REFERENCES

Walters, M. C., Patience, M., Leisenring, W., Eckman, J. R., Scott, J. P., Mentzer, W. C., Davies, S. C., Ohene-Frempong, K., Bernaudin, F., Matthews, D. C., et al. 1996. Bone marrow transplantation for sickle cell disease. N Engl J Med 335:369-376.
2. Giardini, C., and Lucarelli, G. 1999. Bone marrow transplantation for beta-thalassemia. Hematol Oncol Clin North Am 13:1059-1064, viii.
3. May, C., Rivella, S., Callegari, J., Heller, G., Gaensler, K. M., Luzzatto, L., and Sadelain, M. 2000. Therapeutic haemoglobin synthesis in beta-thalassaemic mice expressing lentivirus-encoded human beta-globin. Nature 406:82-86.
4. Pawliuk, R., Westerman, K. A., Fabry, M. E., Payen, E., Tighe, R., Bouhassira, E. E., Acharya, S. A., Ellis, J., London, I. M., Eaves, C. J., et al. 2001. Correction of sickle cell disease in transgenic mouse models by gene therapy. Science 294:2368-2371.
5. Pestina, T. I., Hargrove, P. W., Jay, D., Gray, J. T., Boyd, K. M., and Persons, D. A. 2008. Correction of Murine Sickle Cell Disease Using gamma-Globin Lentiviral Vectors to Mediate High-level Expression of Fetal Hemoglobin. Mol Ther.
6. Bank, A., Dorazio, R., and Leboulch, P. 2005. A phase I/II clinical trial of beta-globin gene therapy for beta-thalassemia. Ann NY Acad Sci 1054:308-316.
7. Cavazzana-Calvo, M., Hacein-Bey, S., de Saint Basile, G., Gross, F., Yvon, E., Nusbaum, P., Selz, F., Hue, C., Certain, S., Casanova, J. L., et al. 2000. Gene therapy of human severe combined immunodeficiency (SCID)-X1 disease. Science 288:669-672.
8. Neven, B., Leroy, S., Decaluwe, H., Le Deist, F., Picard, C., Moshous, D., Mahlaoui, N., Debre, M., Casanova, J. L., Dal Cortivo, L., et al. 2009. Long-term outcome after haematopoietic stem cell transplantation of a single-centre cohort of 90 patients with severe combined immunodeficiency: Long-term outcome of HSCT in SCID. Blood.
9. Ng, E. S., Davis, R. P., Azzola, L., Stanley, E. G., and Elefanty, A. G. 2005. Forced aggregation of defined numbers of human embryonic stem cells into embryoid bodies fosters robust, reproducible hematopoietic differentiation. Blood 106:1601-1603.
10. Lu, S. J., Feng, Q., Caballero, S., Chen, Y., Moore, M. A., Grant, M. B., and Lanza, R. 2007. Generation of functional hemangioblasts from human embryonic stem cells. Nat Methods 4:501-509.
11. Chang, K. H., Nelson, A. M., Fields, P. A., Hesson, J. L., Ulyanova, T., Cao, H., Nakamoto, B., Ware, C. B., and Papayannopoulou, T. 2008. Diverse hematopoietic potentials of five human embryonic stem cell lines. Exp Cell Res 314:2930-2940.
12. Ledran, M. H., Krassowska, A., Armstrong, L., Dimmick, I., Renstrom, J., Lang, R., Yung, S., Santibanez-Coref, M., Dzierzak, E., Stojkovic, M., et al. 2008. Efficient hematopoietic differentiation of human embryonic stem cells on stromal cells derived from hematopoietic niches. Cell Stem Cell 3:85-98.
13. Lu, S. J., Feng, Q., Park, J. S., Vida, L., Lee, B. S., Strausbauch, M., Wettstein, P. J., Honig, G. R., and Lanza, R. 2008. Biologic properties and enucleation of red blood cells from human embryonic stem cells. Blood 112:4475-4484.
14. Ng, E. S., Davis, R. P., Hatzistavrou, T., Stanley, E. G., and Elefanty, A. G. 2008. Directed differentiation of human embryonic stem cells as spin embryoid bodies and a description of the hematopoietic blast colony forming assay. Curr Protoc Stem Cell Biol Chapter 1:Unit 1D 3.
15. Munsie, M. J., Michalska, A. E., O'Brien, C. M., Trounson, A. O., Pera, M. F., and Mountford, P. S. 2000. Isolation of pluripotent embryonic stem cells from reprogrammed adult mouse somatic cell nuclei. Curr Biol 10:989-992.
16. Trounson, A., and Pera, M. 2001. Human embryonic stem cells. Fertil Steril 76:660-661.
17. Yang, X., Smith, S. L., Tian, X. C., Lewin, H. A., Renard, J. P., and Wakayama, T. 2007. Nuclear reprogramming of cloned embryos and its implications for therapeutic cloning. Nat Genet 39:295-302.
18. Takahashi, K., and Yamanaka, S. 2006. Induction of pluripotent stem cells from mouse embryonic and adult fibroblast cultures by defined factors. Cell 126:663-676.
19. Takahashi, K., Tanabe, K., Ohnuki, M., Narita, M., Ichisaka, T., Tomoda, K., and Yamanaka, S. 2007. Induction of pluripotent stem cells from adult human fibroblasts by defined factors. Cell 131:861-872.
20. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. 2007. Induced pluripotent stem cell lines derived from human somatic cells. Science 318:1917-1920.
21. Park, I. H., Zhao, R., West, J. A., Yabuuchi, A., Huo, H., Ince, T. A., Lerou, P. H., Lensch, M. W., and Daley, G. Q. 2008. Reprogramming of human somatic cells to pluripotency with defined factors. Nature 451:141-146.
22. Wernig, M., Meissner, A., Foreman, R., Brambrink, T., Ku, M., Hochedlinger, K., Bernstein, B. E., and Jaenisch, R. 2007. In vitro reprogramming of fibroblasts into a pluripotent ES-cell-like state. Nature 448:318-324.
23. Nakagawa, M., Koyanagi, M., Tanabe, K., Takahashi, K., Ichisaka, T., Aoi, T., Okita, K., Mochiduki, Y., Takizawa, N., and Yamanaka, S. 2008. Generation of induced pluripotent stem cells without Myc from mouse and human fibroblasts. Nat Biotechnol 26:101-106.
24. Maherali, N., Ahfeldt, T., Rigamonti, A., Utikal, J., Cowan, C., and Hochedlinger, K. 2008. A high-efficiency system for the generation and study of human induced pluripotent stem cells. Cell Stem Cell 3:340-345.
25. Liu, H., Zhu, F., Yong, J., Zhang, P., Hou, P., Li, H., Jiang, W., Cai, J., Liu, M., Cui, K., et al. 2008. Generation of induced pluripotent stem cells from adult rhesus monkey fibroblasts. Cell Stem Cell 3:587-590.
26. Feng, B., Jiang, J., Kraus, P., Ng, J. H., Heng, J. C., Chan, Y. S., Yaw, L. P., Zhang, W., Loh, Y. H., Han, J., et al. 2009. Reprogramming of fibroblasts into induced pluripotent stem cells with orphan nuclear receptor Esrrb. Nat Cell Biol.
27. Hanna, J., Wernig, M., Markoulaki, S., Sun, C. W., Meissner, A., Cassady, J. P., Beard, C., Brambrink, T., Wu, L. C., Townes, T. M., et al. 2007. Treatment of sickle cell anemia mouse model with iPS cells generated from autologous skin. Science 318:1920-1923.
28. Varas, F., Stadtfeld, M., De Andres-Aguayo, L., Maherali, N., di Tullio, A., Pantano, L., Notredame, C., Hochedlinger, K., and Graf, T. 2008. Fibroblast derived induced pluripotent stem cells show no common retroviral vector insertions. Stem Cells.
29. Okita, K., Nakagawa, M., Hyenjong, H., Ichisaka, T., and Yamanaka, S. 2008. Generation of mouse induced pluripotent stem cells without viral vectors. Science 322:949-953.
30. Stadtfeld, M., Nagaya, M., Utikal, J., Weir, G., and Hochedlinger, K. 2008. Induced pluripotent stem cells generated without viral integration. Science 322:945-949.
31. Parks, R. J., Chen, L., Anton, M., Sankar, U., Rudnicki, M. A., and Graham, F. L. 1996. A helper-dependent adenovirus vector system: removal of helper virus by Cre-mediated excision of the viral packaging signal. Proc Natl Acad Sci USA 93:13565-13570.
32. Oka, K., and Chan, L. 2005. Construction and characterization of helper-dependent adenoviral vectors for sustained in vivo gene therapy. Methods Mol Med 108:329-350.
33. Parks, R., Evelegh, C., and Graham, F. 1999. Use of helper-dependent adenoviral vectors of alternative serotypes permits repeat vector administration. Gene Ther 6:1565-1573.
34. Groth, A. C., Olivares, E. C., Thyagarajan, B., and Calos, M. P. 2000. A phage integrase directs efficient site-specific integration in human cells. Proc Natl Acad Sci USA 97:5995-6000.
35. Thyagarajan, B., Guimaraes, M. J., Groth, A. C., and Calos, M. P. 2000. Mammalian genomes contain active recombinase recognition sites. Gene 244:47-54.
36. Thyagarajan, B., Olivares, E. C., Hollis, R. P., Ginsburg, D. S., and Calos, M. P. 2001. Site-specific genomic integration in mammalian cells mediated by phage phiC31 integrase. Mol Cell Biol 21:3926-3934.
37. Chalberg, T. W., Genise, H. L., Vollrath, D., and Calos, M. P. 2005. phiC31 integrase confers genomic integration and long-term transgene expression in rat retina. Invest Ophthalmol Vis Sci 46:2140-2146.
38. Chalberg, T. W., Portlock, J. L., Olivares, E. C., Thyagarajan, B., Kirby, P. J., Hillman, R. T., Hoelters, J., and Cabs, M. P. 2006. Integration specificity of phage phiC31 integrase in the human genome. J Mol Biol 357:28-48.
39. Smithies, O., Gregg, R. G., Boggs, S. S., Koralewski, M. A., and Kucherlapati, R. S. 1985. Insertion of DNA sequences into the human chromosomal beta-globin locus by homologous recombination. Nature 317:230-234.
40. Thomas, K. R., and Capecchi, M. R. 1987. Site-directed mutagenesis by gene targeting in mouse embryo-derived stem cells. Cell 51:503-512.
41. Chang, J. C., Ye, L., and Kan, Y. W. 2006. Correction of the sickle cell mutation in embryonic stem cells. Proc Natl Acad Sci USA 103:1036-1040.
42. Jasin, M. 1996. Genetic manipulation of genomes with rare-cutting endonucleases. Trends Genet 12:224-228.
43. Porteus, M. H., and Baltimore, D. 2003. Chimeric nucleases stimulate gene targeting in human cells. Science 300:763.
44. Porteus, M. H., Cathomen, T., Weitzman, M. D., and Baltimore, D. 2003. Efficient gene targeting mediated by adeno-associated virus and DNA double-strand breaks. Mol Cell Biol 23:3558-3565.
45. Zou, J., Maeder, M. L., Mali, P., Pruett-Miller, S. M., Thibodeau-Beganny, S., Chou, B. K., Chen, G., Ye, Z., Park, I. H., Daley, G. Q., et al. 2009. Gene targeting of a disease-related gene in human induced pluripotent stem and embryonic stem cells. Cell Stem Cell 5:97-110.
46. Hockemeyer, D., Soldner, F., Beard, C., Gao, Q., Mitalipova, M., DeKelver, R. C., Katibah, G. E., Amora, R., Boydston, E. A., Zeitler, B., et al. 2009. Efficient targeting of expressed and silent genes in human ESCs and iPSCs using zinc-finger nucleases. Nat Biotechnol 27:851-857.
47. Lombardo, A., Genovese, P., Beausejour, C. M., Colleoni, S., Lee, Y. L., Kim, K. A., Ando, D., Urnov, F. D., Galli, C., Gregory, P. D., et al. 2007. Gene editing in human stem cells using zinc finger nucleases and integrase-defective lentiviral vector delivery. Nat Biotechnol 25:1298-1306.
48. Zhang, X. B., Beard, B. C., Beebe, K., Storer, B., Humphries, R. K., and Kiem, H. P. 2006. Differential effects of HOXB4 on nonhuman primate short- and long-term repopulating cells. PLoS Med 3:e173.
49. Bowles, K. M., Vallier, L., Smith, J. R., Alexander, M. R., and Pedersen, R. A. 2006. HOXB4 overexpression promotes hematopoietic development by human embryonic stem cells. Stem Cells 24:1359-1369.
50. Lu, S. J., Feng, Q., Ivanova, Y., Luo, C., Li, T., Li, F., Honig, G. R., and Lanza, R. 2007. Recombinant HoxB4 fusion proteins enhance hematopoietic differentiation of human embryonic stem cells. Stem Cells Dev 16:547-559.
51. Unger, C., Karner, E., Treschow, A., Stellan, B., Felldin, U., Concha, H., Wendel, M., Hovatta, O., Aints, A., Ahrlund-Richter, L., et al. 2008. Lentiviral-mediated HoxB4 expression in human embryonic stem cells initiates early hematopoiesis in a dose-dependent manner but does not promote myeloid differentiation. Stem Cells 26:2455-2466.
52. Zhang, X. B., Beard, B. C., Trobridge, G. D., Wood, B. L., Sale, G. E., Sud, R., Humphries, R. K., and Kiem, H. P. 2008. High incidence of leukemia in large animals after stem cell gene therapy with a HOXB4-expressing retroviral vector. J Clin Invest 118:1502-1510.
53. Ye, L., Chang, J. C., Lin, C., Sun, X., Yu, J., and Kan, Y. W. 2009. Induced pluripotent stem cells offer new approach to therapy in thalassemia and sickle cell anemia and option in prenatal diagnosis in genetic diseases. Proc Natl Acad Sci USA 106:9826-9830.
54. Dick, J. E., Guenechea, G., Gan, O. I., and Dorrell, C. 2001. In vivo dynamics of human stem cell repopulation in NOD/SCID mice. Ann NY Acad Sci 938:184-190.
55. Ditadi, A., de Coppi, P., Picone, O., Gautreau, L., Smati, R., Six, E., Bonhomme, D., Ezine, S., Frydman, R., Cavazzana-Calvo, M., et al. 2009. Human and murine amniotic fluid c-Kit+Lin-cells display hematopoietic activity. Blood 113:3953-3960.
56. Sands, M. S., and Davidson, B. L. 2006. Gene therapy for lysosomal storage diseases. Mol Ther 13:839-849.
57. Ryan, M. D., and Drew, J. 1994. Foot-and-mouth disease virus 2A oligopeptide mediated cleavage of an artificial polyprotein. Embo J 13:928-933.
58. Donnelly, M. L., Luke, G., Mehrotra, A., Li, X., Hughes, L. E., Gani, D., and Ryan, M. D. 2001. Analysis of the aphthovirus 2A/2B polyprotein ‘cleavage’ mechanism indicates not a proteolytic reaction, but a novel translational effect: a putative ribosomal ‘skip’. J Gen Virol 82:1013-1025.
59. Szymczak, A. L., Workman, C. J., Wang, Y., Vignali, K. M., Dilioglou, S., Vanin, E. F., and Vignali, D. A. 2004. Correction of multi-gene deficiency in vivo using a single ‘self-cleaving’ 2A peptide-based retroviral vector. Nat Biotechnol 22:589-594.
60. Hsiao, E. C., Yoshinaga, Y., Nguyen, T. D., Musone, S. L., Kim, J. E., Swinton, P., Espineda, I., Manalac, C., deJong, P. J., and Conklin, B. R. 2008. Marking embryonic stem cells with a 2A self-cleaving peptide: a NKX2-5 emerald GFP BAC reporter. PLoS ONE 3:e2532.
61. Helms, M. W., Kemming, D., Pospisil, H., Vogt, U., Buerger, H., Korsching, E., Liedtke, C., Schlotter, C. M., Wang, A., Chan, S. Y., et al. 2008. Squalene epoxidase, located on chromosome 8q24. 1, is upregulated in 8q+ breast cancer and indicates poor clinical outcome in stage I and II disease. Br J Cancer 99:774-780.
62. Chang, J. C., Lu, R., Lin, C., Xu, S. M., Kan, Y. W., Porcu, S., Carlson, E., Kitamura, M., Yang, S., Flebbe-Rehwaldt, L., et al. 1998. Transgenic knockout mice exclusively expressing human hemoglobin S after transfer of a 240-kb betas-globin yeast artificial chromosome: A mouse model of sickle cell anemia. Proc Natl Acad Sci USA 95:14886-14890.
63. Johansson, B. M., and Wiles, M. V. 1995. Evidence for involvement of activin A and bone morphogenetic protein 4 in mammalian mesoderm and hematopoietic development. Mol Cell Biol 15:141-151.
64. Choi, K. D., Yu, J., Smuga-Otto, K., Salvagiotto, G., Rehrauer, W., Vodyanik, M., Thomson, J., and Slukvin, I. 2009. Hematopoietic and endothelial differentiation of human induced pluripotent stem cells. Stem Cells 27:559-567.
65. Blelloch, R., Venere, M., Yen, J., and Ramalho-Santos, M. 2007. Generation of induced pluripotent stem cells in the absence of drug selection. Cell Stem Cell 1:245-247.
66. Sommer, C. A., Stadtfeld, M., Murphy, G. J., Hochedlinger, K., Kotton, D. N., and Mostoslavsky, G. 2008. iPS Cell Generation Using a Single Lentiviral Stem Cell Cassette. Stem Cells.
67. Carey, B. W., Markoulaki, S., Hanna, J., Saha, K., Gao, Q., Mitalipova, M., and Jaenisch, R. 2009. Reprogramming of murine and human somatic cells using a single polycistronic vector. Proc Natl Acad Sci USA 106:157-162.
68. Raya, A., Rodriguez-Piza, I., Guenechea, G., Vassena, R., Navarro, S., Barrero, M. J., Consiglio, A., Castella, M., Rio, P., Sleep, E., et al. 2009. Disease-corrected haematopoietic progenitors from Fanconi anaemia induced pluripotent stem cells. Nature 460:53-59.
69. Han, X. D., Lin, C., Chang, J., Sadelain, M., and Kan, Y. W. 2007. Fetal gene therapy of alphathalassemia in a mouse model. Proc Natl Acad Sci USA 104:9007-9011.
70. Shultz, L. D., Lyons, B. L., Burzenski, L. M., Gott, B., Chen, X., Chaleff, S., Kotb, M., Gillies, S. D., King, M., Mangada, J., et al. 2005. Human lymphoid and myeloid cell development in NOD/LtSz-scid IL2R gamma null mice engrafted with mobilized human hemopoietic stem cells. J Immunol 174:6477-6489.
71. Baersch, G., Mollers, T., Hotte, A., Dockhorn-Dworniczak, B., Rube, C., Ritter, J., Jurgens, H., and Vormoor, J. 1997. Good engraftment of B-cell precursor ALL in NOD-SCID mice. Klin Padiatr 209:178-185.
72. Greiner, D. L., Hesselton, R. A., and Shultz, L. D. 1998. SCID mouse models of human stem cell engraftment. Stem Cells 16:166-177.
73. Shultz, L. D., Lang, P. A., Christianson, S. W., Gott, B., Lyons, B., Umeda, S., Leiter, E., Hesselton, R., Wagar, E. J., Leif, J. H., et al. 2000. NOD/LtSz-Rag1null mice: an immunodeficient and radioresistant model for engraftment of human hematolymphoid cells, HIV infection, and adoptive transfer of NOD mouse diabetogenic T cells. J Immunol 164:2496-2507.
74. Shultz, L. D., Banuelos, S., Lyons, B., Samuels, R., Burzenski, L., Gott, B., Lang, P., Leif, J., Appel, M., Rossini, A., et al. 2003. NOD/LtSz-Rag1nullPfpnull mice: a new model system with increased levels of human peripheral leukocyte and hematopoietic stem-cell engraftment. Transplantation 76:1036-1042.
75. Ishikawa, F., Yasukawa, M., Lyons, B., Yoshida, S., Miyamoto, T., Yoshimoto, G., Watanabe, T., Akashi, K., Shultz, L. D., and Harada, M. 2005. Development of functional human blood and immune systems in NOD/SCID/IL2 receptor {gamma} chain(null) mice. Blood 106:1565-1573.
76. Wang, D., Coscoy, L., Zylberberg, M., Avila, P. C., Boushey, H. A., Ganem, D., and DeRisi, J. L. 2002. Microarray-based detection and genotyping of viral pathogens. Proc Natl Acad Sci USA 99:15687-15692.
77. Chiu, C. Y., Alizadeh, A. A., Rouskin, S., Merker, J. D., Yeh, E., Yagi, S., Schnurr, D., Patterson, B. K., Ganem, D., and DeRisi, J. L. 2007. Diagnosis of a critical respiratory illness caused by human metapneumovirus by use of a pan-virus microarray. J Clin Microbiol 45:2340-2343.
78. Chiu, C. Y., Greninger, A. L., Kanada, K., Kwok, T., Fischer, K. F., Runckel, C., Louie, J. K., Glaser, C. A., Yagi, S., Schnurr, D. P., et al. 2008. Identification of cardioviruses related to Theiler's murine encephalomyelitis virus in human infections. Proc Natl Acad Sci USA 105:14124-14129.

What is claimed is:

1. A method comprising:
isolating a fetal somatic cell in utero from a fetus or from cord blood; and
reprogramming said fetal somatic cell to an induced pluripotent stem (iPS) cell.

2. The method of claim 1 wherein said fetal somatic cell comprises a predetermined genetic defect.

3. The method of claim 1 or 2 wherein said reprogramming is with a reprogramming vector comprising an integration sequence and nucleic acid encoding one or more reprogramming factors and an integrase vector comprising nucleic acid encoding an integrase enzyme.

4. The method of claim 3 wherein said reprogramming vector encodes at least Oct4 and Sox2.

5. The method of claim 3 wherein said reprogramming factors are transiently expressed.

6. The method of claim 2 wherein said predetermined genetic defect is corrected in said fetal somatic cell or said iPS cell.

7. The method of claim 1 further comprising transforming said iPS cell to a stem cell.

8. The method of claim 2 further comprising transforming said iPS cell to a stem cell, wherein said predetermined genetic defect is corrected in said stem cell or said iPS cell.

9. The method of claim 6 or 8 wherein said correcting of said predetermined genetic defect comprises transfecting said cell with a correction vector comprising nucleic acid encoding the correction of said predetermined genetic defect, wherein said correction occurs by homologous recombination between the genome of said cell and said correction vector.

10. The method of claim 9 further comprising transfecting said cell with a vector comprising nucleic acid encoding a zinc finger nuclease.

11. The method of claim 1 or 2 wherein said fetal somatic cell is human.

12. The method of claim 11 wherein said human fetal somatic cell is isolated from the amniotic fluid or the chorionic villus of a pregnant human female.

13. The method of claim 1 wherein said fetus is diagnosed with a predetermined genetic defect prior to said isolating of said fetal somatic cell.

14. The method of claim 13 wherein said isolated fetal cells are used for prenatal diagnosis of said genetic defect and as a source of cells for said reprogramming or for correcting said predetermined genetic defect.

15. The method of claim 13 wherein said predetermined genetic defect causes sickle cell disease, beta-thalassemia, cystic fibrosis, Tay-Sachs disease, adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), juvenile-onset, type 1 diabetes mellitus (JDM), Down syndrome (DS)/trisomy 21, mucopolysaccharidosis and Lesch-Nyhan syndrome.

16. The method of claim 15 wherein said predetermined genetic defect causes sickle cell disease or beta-thalassemia.

17. The method of claim 7 further comprising transplanting said stem cell in utero to said fetus or to the individual that develops from said fetus.

18. The method of claim 8 further comprising transplanting said stem cell in utero to said fetus or to the individual that develops from said fetus to treat the disease caused by said genetic defect.

19. An iPS cell made according to the method of claim 1, 2, 3 or 6.

20. A stem cell made according to the method of claim 7 or 8.
